SEQUENCE LISTING (23 sequences)

SEQ ID NO: 1 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 10 aa, Artificial Sequence
  Template 1 loop insertion sequence
STWFDGGYAT

SEQ ID NO: 3 - protein, 10 aa, Artificial Sequence
  Template 1.1 loop insertion sequence
STWFIGGYAT

SEQ ID NO: 4 - protein, 10 aa, Artificial Sequence
  Template 7.1 loop insertion sequence
GLDHRGKGYV

SEQ ID NO: 5 - protein, 10 aa, Artificial Sequence
  Template 1.2 loop insertion sequence
STWFDKGYAT

SEQ ID NO: 6 - protein, 101 aa, Homo sapiens
CCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKS

SEQ ID NO: 7 - protein, 101 aa, Homo sapiens
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKS

SEQ ID NO: 8 - protein, 108 aa, Homo sapiens
ECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREP

SEQ ID NO: 9 - protein, 108 aa, Homo sapiens
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 10 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 11 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 12 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 13 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 14 - protein, 106 aa, Homo sapiens
DQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPK

SEQ ID NO: 15 - protein, 111 aa, Homo sapiens
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLA

SEQ ID NO: 16 - protein, 111 aa, Homo sapiens; identical to SEQ ID NO: 15

SEQ ID NO: 17 - protein, 108 aa, Homo sapiens
AAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVS

SEQ ID NO: 18 - protein, 108 aa, Homo sapiens
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNP

SEQ ID NO: 19 - protein, 105 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 20 - protein, 105 aa, Homo sapiens
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 21 - protein, 105 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSP

SEQ ID NO: 22 - protein, 105 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 23 - protein, 110 aa, Homo sapiens
GVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKST

(19) United States
(12) Patent Application Publication
(10) Pub. No.: US 2023/0303715 A1
(43) Pub. Date: Sep. 28, 2023

(54) IMMUNOGLOBULIN Fc REGION VARIANTS COMPRISING STABILITY-ENHANCING MUTATIONS

(71) Applicant: Zymeworks BC Inc., Vancouver (CA)

(72) Inventors: Genevieve DESJARDINS, Vancouver, BC (CA); Eric ESCOBAR-CABRERA, Vancouver, BC (CA); Antonios SAMIOTAKIS, Vancouver, BC (CA); Gavin Carl JONES, Middlesex (GB)

(21) Appl. No.: 17/999,477

(22) PCT Filed: May 20, 2021
(86) PCT No.: PCT/CA2021/050691

§ 371 Date: Nov. 21, 2022

Related U.S. Application Data
(60) Provisional application No. 63/027,569, filed on May 20, 2020.
(60) Provisional application No. 63/163,686, filed on Mar. 19, 2021.

(51) Int. Cl. C07K 16/32; C12N 15/63

(52) U.S. Cl. CPC C07K 16/32; C12N 15/63; C07K 2317/524; C07K 2317/55; C07K 2317/622

(57) ABSTRACT

Fc variants are described comprising one or more amino acid mutations that increase the stability of the Fc variant as compared to a parental Fc that does not include the one or more amino acid mutations, as well as polypeptides comprising an Fc variant and polynucleotides encoding an Fc variant.

### FIELD

The present disclosure relates to the field of immunoglobulins and, in particular, to immunoglobulin Fc variants comprising stability-enhancing mutations.

### BACKGROUND

Immunoglobulin-based drugs are becoming an increasingly important therapeutic approach with monoclonal antibodies having been identified as the predominant treatment modality for various diseases over the past 25 years.

Considerable research has been directed to engineering immunoglobulins to improve various functions. For example, modifications have been made to antibody Fc regions in order to improve pharmacokinetics, enhance or reduce antibody-dependent cellular cytotoxicity (ADCC) activity, enhance or reduce selectivity for specific Fcγ receptors or the FcRn receptor, or improve formation of heterodimeric Fc regions in bispecific antibodies. Such modifications, however, can have adverse effects on other properties of the antibody, including thermostability.

Efforts to improve the stability of engineered antibodies include the introduction of mutations to provide new disulphide bonds (Gong et al., 2009, J Biol Chem, 284(21):14203-14210; Jacobsen et al., 2017, J Biol Chem, 292(5):1865-1875) and introduction of combinations of point mutations (International Patent Application Publication No. WO 2012/032080). Methods for improving stability of an antibody Fc region by introducing various amino acid substitutions to a loop region of the antibody Fc region have also been described (U.S. Patent Application Publication No. 2015/0210763).

This background information is provided for the purpose of making known information believed by the applicant to be of possible relevance to the present disclosure. No admission is necessarily intended, nor should be construed, that any of the preceding information constitutes prior art against the claimed invention.

### SUMMARY

Described herein are immunoglobulin Fc region variants comprising stability-enhancing mutations. One aspect of the present disclosure relates to an Fc variant comprising one or more stability-enhancing amino acid mutations selected from: a mutation at position 250, where the mutation is a substitution of the amino acid at position 250 with Ala, Ile or Val; a mutation at position 287, where the mutation is a substitution of the amino acid at position 287 with Phe, His, Met, Trp or Tyr; a mutation at position 308, where the mutation is a substitution of the amino acid at position 308 with Ile; a mutation at position 309, where the mutation is a substitution of the amino acid at position 309 with Gln or Thr; a mutation at position 428, where the mutation is a substitution the amino acid at position 428 with Phe, and a pair of mutations at position 242 and position 336, where both mutations are substitutions with Cys, wherein the Fc variant has an increased CH2 domain melting temperature (Tm) as compared to a parental Fc that does not include the one or more stability-enhancing amino acid mutations.

Another aspect of the present disclosure relates to an Fc variant comprising from one to three stability-enhancing amino acid mutations, the mutations comprising: (a) one or more mutation selected from: a mutation at position 287 which is a substitution with Phe, His, Met, Trp or Tyr; a mutation at position 308 which is a substitution with Ile, and a mutation at position 309 which is a substitution with Gln or Thr, or (b) two or more mutations selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr; a mutation at position 428 which is a substitution with Phe, and a pair of mutations at position 242 and position 336 which are both substitutions with Cys, or (c) three or more mutations comprising: a pair of mutations at position 242 and position 336 which are both substitutions with Cys, and a mutation selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr, and a mutation at position 428 which is a substitution with Phe, wherein the Fc variant has an increased CH2 domain melting temperature (Tm) as compared to a parental Fc that does not include the one or more stability-enhancing amino acid mutations.

Another aspect of the present disclosure relates to a polypeptide comprising the Fc variant as described herein and one or more proteinaceous moieties fused or covalently attached to the Fc variant.

Another aspect of the present disclosure relates to a polynucleotide or set of polynucleotides encoding an Fc variant as described herein.

Another aspect of the present disclosure relates to a polynucleotide or set of polynucleotides encoding a polypeptide comprising the Fc variant as described herein and one or more proteinaceous moieties fused or covalently attached to the Fc variant.

Another aspect of the present disclosure relates to a vector or set of vectors comprising one or more polynucleotides encoding an Fc variant as described herein.

Another aspect of the present disclosure relates to a vector or set of vectors comprising one or more polynucleotides encoding a polypeptide comprising the Fc variant as described herein and one or more proteinaceous moieties fused or covalently attached to the Fc variant.

Another aspect of the present disclosure relates to a host cell comprising one or more polynucleotides encoding an Fc variant as described herein.

Another aspect of the present disclosure relates to a host cell comprising one or more polynucleotides encoding a polypeptide comprising the Fc variant as described herein and one or more proteinaceous moieties fused or covalently attached to the Fc variant.

Another aspect of the present disclosure relates to a method of preparing an Fc variant as described herein, the method comprising transfecting a host cell with one or more polynucleotides encoding the Fc variant, and culturing the host cell under conditions suitable for expression of the Fc variant.

Another aspect of the present disclosure relates to a method of preparing a polypeptide comprising the Fc variant as described herein and one or more proteinaceous moieties fused or covalently attached to the Fc variant, the method comprising transfecting a host cell with one or more polynucleotides encoding the polypeptide, and culturing the host cell under conditions suitable for expression of the polypeptide.

Another aspect of the present disclosure relates to a pharmaceutical composition comprising an Fc variant as described herein.

Another aspect of the present disclosure relates to a pharmaceutical composition comprising a polypeptide comprising the Fc variant as described herein and one or more proteinaceous moieties fused or covalently attached to the Fc variant.

Another aspect of the present disclosure relates to a method of increasing the CH2 domain melting temperature (Tm) of an Fc comprising introducing into a parental Fc one or more stability-enhancing amino acid mutations to provide an Fc variant having an increased CH2 domain Tm as compared to the parental Fc, the mutations selected from: a mutation at position 250, where the mutation is a substitution of the amino acid at position 250 with Ala, Ile or Val; a mutation at position 287, where the mutation is a substitution of the amino acid at position 287 with Phe, His, Met, Trp or Tyr; a mutation at position 308, where the mutation is a substitution of the amino acid at position 308 with Ile; a mutation at position 309, where the mutation is a substitution of the amino acid at position 309 with Gln or Thr; a mutation at position 428, where the mutation is a substitution the amino acid at position 428 with Phe, and a pair of mutations at position 242 and position 336, where both mutations are substitutions with Cys.

Another aspect of the present disclosure relates to a method of increasing the CH2 domain melting temperature (Tm) of an Fc comprising introducing into a parental Fc one to three stability-enhancing amino acid mutations to provide an Fc variant having an increased CH2 domain Tm as compared to the parental Fc, the mutations comprising: (a) one or more mutation selected from: a mutation at position 287 which is a substitution with Phe, His, Met, Trp or Tyr; a mutation at position 308 which is a substitution with Ile, and a mutation at position 309 which is a substitution with Gln or Thr, or (b) two or more mutations selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr; a mutation at position 428 which is a substitution with Phe, and a pair of mutations at position 242 and position 336 which are both substitutions with Cys, or (c) three or more mutations comprising: a pair of mutations at position 242 and position 336 which are both substitutions with Cys, and a mutation selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr, and a mutation at position 428 which is a substitution with Phe.

### BRIEF DESCRIPTION OF THE DRAWINGS

FIG. 1 provides (A) the sequence of the IgG1 Fc region sequence [SEQ ID NO:1], and (B) a structural view of the IgG1 Fc region (PDB ID: 4BSV) showing the locations of the exemplary stability-enhancing designs T250V, A287F and M428F.

FIG. 2 shows the improvement in CH2 domain melting temperature (Tm) resulting from introducing exemplary stability-enhancing mutations into various Fc scaffolds (A) Scaffold 3 comprising asymmetrical mutations to promote heterodimeric Fc formation; (B) Scaffold 6 comprising N297A mutation, and (C) Scaffold 7 comprising S239D/I332E mutations. Scaffold 1 is a homodimeric IgG1 Fc.

FIG. 3 shows (A) a sequence alignment of the CH2 domains of IgA, IgD and IgG with the CH3 domains of IgE and IgM, and (B) a sequence alignment of the CH3 domains of IgA, IgD and IgG with the CH4 domains of IgE and IgM. Positions equivalent to IgG1 T250, A287 and M428 are boxed.

FIG. 4 shows the correlation between aggregation induced by incubation at 40° C. for 2 weeks under acidic or neutral conditions and the thermal stability of the CH2 domain for antibody variants with and without the stability-enhancing mutations T250V/A287F, (A) standard scale x-axis, incubation under mildly acidic conditions, (B) standard scale x-axis, incubation under neutral conditions, and (C) log scale x-axis, incubation under mildly acidic conditions, and (D) log scale x-axis, incubation under neutral conditions. Variants showing a small or no negative change in aggregation through the study are omitted. Parental sequences (non-stabilized) are indicated by circles, stabilized variants are indicated by squares, with each of the non-stabilized and corresponding stabilized variants connected by an arrow.

### DETAILED DESCRIPTION

Described herein are Fc variants comprising one or more amino acid mutations that increase the stability of the Fc variant as compared to a parental Fc that does not include the one or more amino acid mutations. These mutations are referred to herein as “stability-enhancing amino acid mutations” or “stability-enhancing mutations.”

In certain embodiments, the one or more stability-enhancing amino acid mutations comprised by the Fc variant are selected from:
a mutation at position 250, where the mutation is a substitution of the amino acid at position 250 with Ala, Ile or Val;
a mutation at position 287, where the mutation is a substitution of the amino acid at position 287 with Phe, His, Met, Trp or Tyr;
a mutation at position 308, where the mutation is a substitution of the amino acid at position 308 with Ile;
a mutation at position 309, where the mutation is a substitution of the amino acid at position 309 with Gln or Thr;
a mutation at position 428, where the mutation is a substitution of the amino acid at position 428 with Phe, and
a mutation at position 242 and a mutation at position 336, where both mutations are substitutions with Cys.

Certain embodiments of the present disclosure relate to polypeptides comprising an Fc variant as described herein. Examples of such polypeptides include, but are not limited to, antibodies, antibody fragments and Fc fusion proteins. Polypeptides comprising an Fc variant as described herein may find use as therapeutics, diagnostics or research tools.

Certain embodiments of the present disclosure relate to polynucleotides encoding the Fc variants described herein and polynucleotides encoding the polypeptides comprising the Fc variants, as well as host cells comprising the polynucleotides and methods of using the polynucleotides and host cells to prepare the Fc variants or polypeptides comprising the Fc variants.

Certain embodiments of the present disclosure relate to methods of stabilizing an Fc (the parental Fc) by introducing one or more stability-enhancing mutations described herein into the Fc. Some embodiments of the present disclosure relate to methods of increasing the CH2 domain melting temperature (Tm) of an Fc (the parental Fc) by introducing one or more stability-enhancing mutations described herein into the Fc. The parental Fc may be a wild-type Fc or it may itself be a variant Fc that already includes one or more amino acid mutations, for example, to improve a function of the Fc region.

### Definitions

Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

The term “isolated,” as used herein with reference to a material, means that the material is removed from its original environment (for example, the natural environment if it is naturally occurring). For example, a naturally occurring polynucleotide or polypeptide present in a living animal is not isolated, but the same polynucleotide or polypeptide separated from some or all of the co-existing materials in the natural system, is isolated. Such polynucleotides could be part of a vector and/or such polynucleotides or polypeptides could be part of a composition, and still be isolated in that such vector or composition is not part of its natural environment.

The terms “Fc region” and “Fc,” as used interchangeably herein, refer to a C-terminal region of an immunoglobulin heavy chain. The human IgG heavy chain Fc region sequence, for example, is usually defined as extending from position 239 to the C-terminus of the heavy chain. An “Fc polypeptide” of a dimeric Fc refers to one of the two polypeptides forming the dimeric Fc domain, i.e. a polypeptide comprising C-terminal constant regions of an immunoglobulin heavy chain that is capable of stable self-association. An Fc region typically comprises a CH2 domain and a CH3 domain. The Fc region may also be considered to encompass the hinge region in certain embodiments.

The “CH2 domain” of a human IgG Fc region is typically defined as extending from position 239 to position 340. The “CH3 domain” is typically defined as comprising the amino acids residues C-terminal to the CH2 domain in an Fc region, i.e. from position 341 to position 447. The “hinge region” of human IgG1 is generally defined as extending from position 216 to position 238 (Burton, 1985, Molec. Immunol., 22:161-206). Hinge regions of other IgG isotypes may be aligned with the IgG1 sequence by aligning the first and last cysteine residues that form inter-heavy chain disulfide bonds.

Unless otherwise specified herein, numbering of amino acid residues in the Fc region is according to the EU numbering system, also called the EU index, as described in Kabat et al, Sequences of Proteins of Immunological Interest, 5th Ed. Public Health Service, National Institutes of Health, Bethesda, MD (1991).

Naturally-occurring amino acids are identified throughout by the conventional three- or one-letter abbreviations indicated in Table A below, as generally accepted in the art and recommended by the IUPAC-IUB commission in biochemical nomenclature.

TABLE A
Amino acid codes

| Name | 3-Letter Code | 1-Letter Code |
|---|---|---|
| Alanine | Ala | A |
| Arginine | Arg | R |
| Asparagine | Asp | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

It is to be understood that the positive recitation of a feature in one embodiment, serves as a basis for excluding the feature in an alternative embodiment. In particular, where a list of options is presented for a given embodiment or claim, it is to be understood that one or more option may be deleted from the list and the shortened list may form an alternative embodiment, whether or not such an alternative embodiment is specifically referred to.

It is contemplated that any embodiment disclosed herein with respect to an Fc variant can be implemented with respect to a method, use or composition disclosed herein, and vice versa.

### Fc Variants

The Fc variants of the present disclosure comprise one or more amino acid mutations (“stability-enhancing mutations”) that increase the stability of the Fc variant as compared to the parental Fc. The increased stability may result in increased thermostability of the CH2 domain, decreased likelihood of aggregation, increased serum half-life, improved manufacturability, or a combination thereof. In certain embodiments, the one or more stability-enhancing mutations comprised by the Fc variant increase the thermostability (Tm) of the CH2 domain as compared to the parental Fc.

In some embodiments, the one or more stability-enhancing mutations comprised by the Fc variant increase the thermostability (Tm) of the CH2 domain as compared to the parental Fc and also decrease aggregation of the Fc region. In some embodiments, the one or more stability-enhancing mutations comprised by the Fc variant increase the thermostability (Tm) of the CH2 domain as compared to the parental Fc and decrease aggregation of the Fc region at low pH. “Low pH” in this context refers to a pH between about 4.0 and 7.5.

In some embodiments, the one or more stability-enhancing mutations comprised by the Fc variant increase the thermostability (Tm) of the CH2 domain as compared to the parental Fc and decrease aggregation of the Fc region under mildly acidic conditions, where the mildly acidic conditions comprise a pH below neutral. In some embodiments, the mildly acidic conditions comprise a pH between about 4.0 and 7.0. In some embodiments, the mildly acidic conditions comprise a pH between about 4.0 and 6.5.

The Fc variant may comprise one stability-enhancing mutation, or it may comprise more than one stability-enhancing mutation. In certain embodiments, the Fc variant comprises between one and five stability-enhancing mutations. In some embodiments, the Fc variant comprises between one and four stability-enhancing mutations. In some embodiments, the Fc variant comprises between one and three stability-enhancing mutations. In some embodiments, the Fc variant comprises 1, 2 or 3 stability-enhancing mutations.

In certain embodiments, the CH2 domain Tm of the Fc variant is increased by at least 0.5° C. as compared to the parental Fc. In some embodiments, the CH2 domain Tm of the Fc variant is increased by at least 1.0° C., at least 1.5° C., at least 2.0° C., at least 2.5° C. or at least 3.0° C., as compared to the parental Fc. In some embodiments, the CH2 domain Tm of the Fc variant is increased by at least 5.0° C., at least 5.5° C., at least 6.0° C., at least 6.5° C. or at least 7.0° C., as compared to the parental Fc.

In certain embodiments, the CH2 domain Tm of the Fc variant is increased by between about 0.5° C. and about 6.5° C. as compared to the parental Fc. In some embodiments, the CH2 domain Tm of the Fc variant is increased by between about 0.5° C. and about 9.0° C. as compared to the parental Fc. In some embodiments, the CH2 domain Tm of the Fc variant is increased by between about 1.0° C. and about 9.0° C., between about 2.0° C. and about 9.0° C., or between about 3.0° C. and about 9.0° C., as compared to the parental Fc. In some embodiments, the CH2 domain Tm of the Fc variant is increased by between about 2.0° C. and about 10.5° C., or between about 3.0° C. and about 10.5° C., as compared to the parental Fc.

In certain embodiments, the CH2 domain Tm is measured by DSC or DSF.

The parental Fc may be a wild-type Fc or it may itself be a variant Fc that already includes one or more amino acid mutations, for example, to improve a function of the Fc region. In some embodiments, the parental Fc may be an Fc that includes one or more amino acid mutations that functionally enhance the Fc region. In some embodiments, the parental Fc may be an Fc that includes one or more amino acid mutations that functionally enhance the Fc region but result in a decrease in stability as compared to a wild-type Fc. In some embodiments, the parental Fc may comprise one or more amino acid mutations that functionally enhance the Fc region but decrease the thermostability of the CH2 domain as compared to a wild-type Fc.

Examples of amino acid mutations that functionally enhance the Fc region but decrease the thermostability of the CH2 domain as compared to a wild-type Fc include, but are not limited to, mutations that promote heterodimeric Fc formation (such as knobs-into-holes or electrostatic steering mutations described by Atwell et al., 1997, J Biol Chem, 270:26-35 and Gunasekaran et al., 2010, J Biol Chem, 285(25):19637-19646), mutations that produce an aglycosylated Fc (such as the N297A mutation described in Lund et al., 1995, FASEB, 9(1):115-119; Leabman et al., 2013, mAbs, 5(6):896-903 and Jacobsen et al., 2017, JBC, 292(5):1865-1875) and mutations that alter FcγR selectivity (such as the S239D/I332E or S239D/A330L/I332E mutations which increase affinity for FcγRIIIa as described by Lazar et al., 2006, PNAS, 103(11):4005-4010 and Oganesyan et al., 2008, Molec Immunol, 45(7):1872-1882, or the E233D/G237D/P238D/H268D/P271G/A330R mutations which increase selectivity for FcγRIIb as described by Mimoto, et al., 2013, Protein Eng. Des. Sel., 26:589-598). Additional examples of amino acid mutations that functionally enhance the Fc region but decrease the thermostability of the CH2 domain as compared to a wild-type or parental Fc are described in the Examples herein.

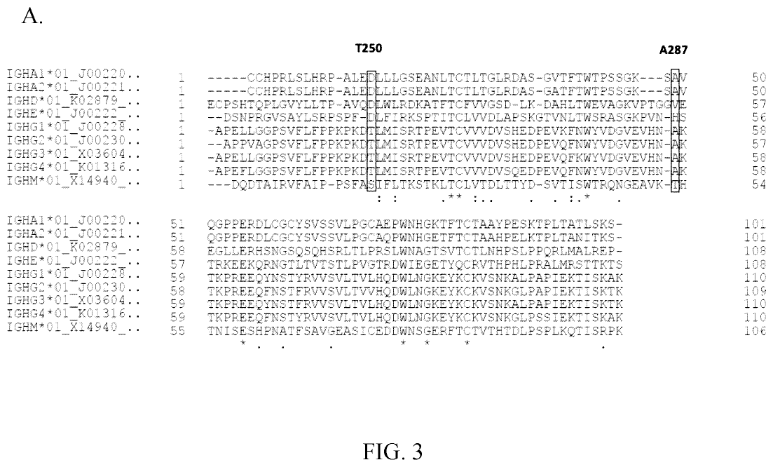

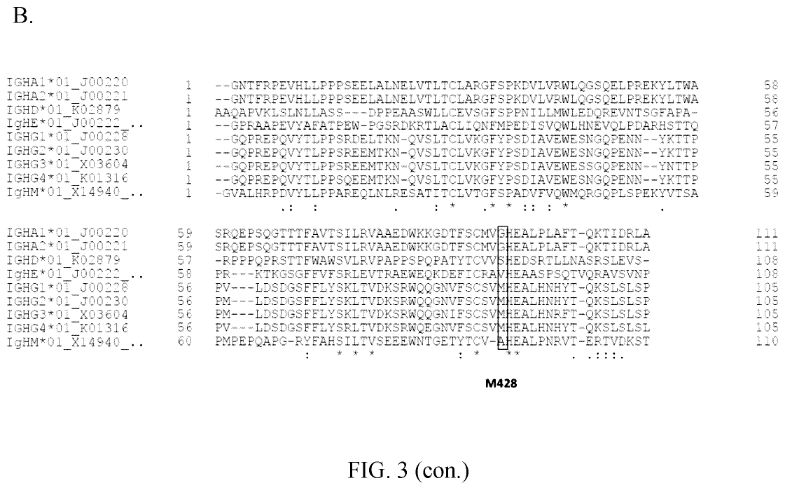

The parental Fc into which the stability-enhancing mutations are introduced may be an IgG Fc, an IgA Fc, an IgD Fc, an IgE Fc or an IgM Fc. While the amino acid numbering used herein relates to an IgG Fc, one skilled in the art could readily determine the equivalent positions for the mutations in other Ig Fc sequences by sequence alignment using one of a number of sequence alignment tools known in the art. Accordingly, reference herein to a specific position in the Fc region for a stability-enhancing mutation is intended to encompass the specified position in an IgG Fc, as well as the corresponding position in an IgA, IgD, IgE or IgM Fc region. A sequence alignment of the CH2 domains of IgA, IgD and IgG with the CH3 domains of IgE and IgM, and a sequence alignment of the CH3 domains of IgA, IgD and IgG with the CH4 domains of IgE and IgM are shown in FIGS. 3A and 3B.

In certain embodiments, the Fc variant is based on an IgG, IgA, IgD, IgE or IgM Fc. In some embodiments, the Fc variant is based on a human IgG, IgA, IgD, IgE or IgM Fc. In some embodiments, the Fc variant is based on an IgG or IgA Fc. In some embodiments, the Fc variant is based on a human IgG or IgA Fc. In some embodiments, the Fc variant is based on an IgG Fc. In some embodiments, the Fc variant is based on a human IgG Fc.

In certain embodiments, the Fc variant is based on an IgG Fc, which may be an IgG1, IgG2, IgG3 or IgG4 Fc. In some embodiments, the Fc variant is based on a human IgG1, IgG2, IgG3 or IgG4 Fc. A sequence alignment of the human IgG1, IgG2, IgG3 and IgG4 CH2 and CH3 domains is provided in FIGS. 3A and 3B. In some embodiments, the Fc variant is based on an IgG1 Fc. In some embodiments, the Fc variant is based on a human IgG1 Fc.

### Stability-Enhancing Mutations

As described herein, in silico and bioinformatic approaches were employed to identify positions within the Fc region that could be mutated in order to improve stability of the Fc. These approaches identified the mutations shown in Table 1 as stability-enhancing mutations.

TABLE 1
Stability-Enhancing Mutations

| Position (EU) | Mutation |
|---|---|
| 250 | T250A, T250I, T250V |
| 287 | A287F, A287H, A287M, A287W, A287Y |
| 308 | V308I |
| 309 | L309Q, L309T |
| 428 | M428F |
| 240 & 332 | V240C_I332C |
| 242 & 336 | L242C_I336C |

In particular, in silico approaches identified the following amino acid mutations as mutations that increase stability of an Fc when introduced as single mutations:
a mutation at position 287 selected from A287F, A287H, A287M, A287W and A287Y, and
the mutation M428F,

and bioinformatics approaches identified the following amino acid mutations as mutations that increase stability of an Fc when introduced as single mutations:
a mutation at position 250 selected from T250A, T250I and T250V;
the mutation V308I, and
a mutation at position 309 selected from L309Q and L309T.

In addition, the mutation pairs L242C_I336C, V240C_I332C and V263C_V302C, each of which introduce an additional disulphide bond into the Fc region, were shown to increase stability of the Fc in the absence of any additional stability-enhancing mutations.

Combinations of the stability-enhancing mutations shown in Table 1 were also shown to further increase the stability of the Fc variant.

Accordingly, in certain embodiments, the Fc variant comprises one or more stability-enhancing mutations of which at least one mutation is selected from the mutations shown in Table 1. In same embodiments, the Fc variant comprises one or more stability-enhancing mutations selected from the mutations shown in Table 1.

In some embodiments, the Fc variant comprises one or more stability-enhancing mutations of which at least one mutation is selected from:
a mutation at position 287, where the mutation is a substitution of the amino acid at position 287 with Phe, His, Met, Trp or Tyr;
a mutation at position 308, where the mutation is a substitution of the amino acid at position 308 with Ile, and
a mutation at position 309, where the mutation is a substitution of the amino acid at position 309 with Gln or Thr.

In certain embodiments, the Fc variant comprises a single stability-enhancing mutation. In some embodiments, the Fc variant comprises a single stability-enhancing mutation selected from:
a mutation at position 287, where the mutation is a substitution of the amino acid at position 287 with Phe, His, Met, Trp or Tyr;
a mutation at position 308, where the mutation is a substitution of the amino acid at position 308 with Ile, and
a mutation at position 309, where the mutation is a substitution of the amino acid at position 309 with Gln or Thr.

In some embodiments, the mutation at position 287 comprised by the Fc variant is a substitution of the amino acid at position 287 with Phe. In some embodiments, the mutation at position 309 comprised by the Fc variant is a substitution of the amino acid at position 309 with Gln.

In certain embodiments, the Fc variant comprises a pair of stability-enhancing mutations each of which introduces a cysteine residue allowing for formation of a new disulphide bond in the Fc region. In some embodiments, the pair of stability-enhancing mutations is selected from: 242C_336C, 240C_332C and 263C_302C. In some embodiments, the pair of stability-enhancing mutations is 242C_336C or 240C_332C. In some embodiments, the pair of stability-enhancing mutations is 242C_336C.

In certain embodiments, the Fc variant comprises two or more stability-enhancing mutations. In some embodiments, the Fc variant comprises two or more stability-enhancing mutations selected from:
a mutation at position 250, where the mutation is a substitution of the amino acid at position 250 with Ala, Ile or Val;
a mutation at position 287 where the mutation is a substitution of the amino acid at position 287 with Phe, His, Met, Trp or Tyr;
a mutation at position 308, where the mutation is a substitution of the amino acid at position 308 with Ile;
a mutation at position 309, where the mutation is a substitution of the amino acid at position 309 with Gln or Thr;
a mutation at position 428, where the mutation is substitution of the amino acid at position 428 with Phe, and
a pair of mutations at position 242 and position 336, where each of the mutations is a substitution with Cys.

In certain embodiments, the Fc variant comprises two stability-enhancing mutations. In some embodiments, the Fc variant comprises:
two stability-enhancing mutations selected from: a mutation at position 250, a mutation at position 287, a mutation at position 308, a mutation at position 309 and a mutation at position 428, where the mutation at position 250 is a substitution with Ala, Ile or Val; the mutation at position 287 is a substitution with Phe, His, Met, Trp or Tyr; the mutation at position 308 is a substitution with Ile; the mutation at position 309 is a substitution with Gln or Thr, and the mutation at position 428 is a substitution with Phe, or a pair of mutations at position 242 and position 336, where each of the mutations is a substitution with Cys.

In some embodiments, the Fc variant comprises:
a mutation at position 250 and a mutation at position 287, where the mutation at position 250 is a substitution with Ala, Ile or Val, and the mutation at position 287 is a substitution with Phe, His, Met, Trp or Tyr;
a mutation at position 250 and a mutation at position 308, where the mutation at position 250 is a substitution with Ala, Ile or Val, and the mutation at position 308 is a substitution with Ile;
a mutation at position 250 and a mutation at position 309, where the mutation at position 250 is a substitution with Ala, Ile or Val, and the mutation at position 309 is a substitution with Gln or Thr;
a mutation at position 250 and a mutation at position 428, where the mutation at position 250 is a substitution with Ala, Ile or Val, and the mutation at position 428 is a substitution with Phe;
a mutation at position 287 and a mutation at position 308, where the mutation at position 287 is a substitution with Phe, His, Met, Trp or Tyr, and the mutation at position 308 is a substitution with Ile;
a mutation at position 287 and a mutation at position 309, where the mutation at position 287 is a substitution with Phe, His, Met, Trp or Tyr, and the mutation at position 309 is a substitution with Gln or Thr;
a mutation at position 287 and a mutation at position 428, where the mutation at position 287 is a substitution with Phe, His, Met, Trp or Tyr, and the mutation at position 428 is a substitution with Phe;
a mutation at position 308 and a mutation at position 309, where the mutation at position 308 is a substitution with Ile, and the mutation at position 309 is a substitution with Gln or Thr;
a mutation at position 308 and a mutation at position 428, where the mutation at position 308 is a substitution with Ile, and the mutation at position 428 is a substitution with Phe;
a mutation at position 309 and a mutation at position 428, where the mutation at position 309 is a substitution with Gln or Thr, and the mutation at position 428 is a substitution with Phe, or
a pair of mutations at position 242 and position 336, where each of the mutations is a substitution with Cys.

In some embodiments, the Fc variant comprises:
a mutation at position 250 and a mutation at position 287, where the mutation at position 250 is a substitution with Ala, Ile or Val, and the mutation at position 287 is a substitution with Phe, His, Met, Trp or Tyr;
a mutation at position 250 and a mutation at position 309, where the mutation at position 250 is a substitution with Ala, Ile or Val, and the mutation at position 309 is a substitution with Gln or Thr;
a mutation at position 250 and a mutation at position 428, where the mutation at position 250 is a substitution with Ala, Ile or Val, and the mutation at position 428 is a substitution with Phe;
a mutation at position 287 and a mutation at position 428, where the mutation at position 287 is a substitution with Phe, His, Met, Trp or Tyr, and the mutation at position 428 is a substitution with Phe, or
a pair of mutations at position 242 and position 336, where each of the mutations is a substitution with Cys.

In some embodiments, the mutation at position 250 comprised by the Fc variant is a substitution with Val. In some embodiments, the mutation at position 287 comprised by the Fc variant is a substitution with Phe. In some embodiments, the mutation at position 309 comprised by the Fc variant is a substitution with Gln.

In some embodiments, the Fc variant comprises the stability-enhancing mutations 250V/287F, 250V/308I, 250V/309Q, 250V/428F, 287F/308I, 287F/309Q, 287F/428F, 308I/309Q, 308I/428F, 309Q/428F or 242C/336C.

In some embodiments, the Fc variant comprises the stability-enhancing mutations 250V/287F, 250V/309Q, 250V/428F, 287F/428F or 242C_336C.

In some embodiments, the Fc variant comprises the stability-enhancing mutations 250V/287F, 250V/309Q, 250V/428F or 287F/428F.

In some embodiments, the stability-enhancing mutations comprised by the Fc variant are selected from: 250V, 287F, 308I, 309Q, 428F, 242C_336C, 287F/428F, 250V/287F, 250V/309Q, 250V/428F and 242C_336C/308I.

In some embodiments, the stability-enhancing mutations comprised by the Fc variant are selected from: 287F, 308I, 309Q, 242C_336C, 287F/428F, 250V/287F, 250V/309Q, 250V/428F and 242C_336C/308I.

In certain embodiments, the Fc variant comprises three or more stability-enhancing mutations. In some embodiments, the Fc variant comprises three or more stability-enhancing mutations selected from:
a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr; a mutation at position 428 which is a substitution with Phe, and a pair of mutations at position 242 and position 336 which are both substitutions with Cys.

In some embodiments, the Fc variant comprises three stability-enhancing mutations. In some embodiments, the Fc variant comprises three stability-enhancing mutations selected from:
a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr; a mutation at position 428 which is a substitution with Phe, and a pair of mutations at position 242 and position 336 which are both substitutions with Cys.

In some embodiments, the Fc variant comprises:
a pair of mutations at position 242 and position 336 which are both substitutions with Cys, and
a mutation selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr, and a mutation at position 428 which is a substitution with Phe.

In certain embodiments, the Fc variant comprises between one and three stability-enhancing mutations. In some embodiments, the Fc variant comprises:
one or more mutations selected from: a mutation at position 287 which is a substitution with Phe, His, Met, Trp or Tyr; a mutation at position 308 which is a substitution with Ile, and a mutation at position 309 which is a substitution with Gln or Thr, or
two or more mutations selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr; a mutation at position 428 which is a substitution with Phe, and a pair of mutations at position 242 and position 336 which are both substitutions with Cys, or
three or more mutations comprising: a mutation at position 242 and a mutation at position 336 which are both substitutions with Cys, and a mutation selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr, and a mutation at position 428 which is a substitution with Phe.

In certain embodiments, the Fc variant is an IgG Fc variant. In certain embodiments, the IgG Fc variant comprises one or more stability-enhancing mutations. In some embodiments, the IgG Fc variant comprises one or more stability-enhancing mutations of which at least one mutation is selected from:
a mutation at position 287 selected from A287F, A287H, A287M, A287W and A287Y;
the mutation V308I, and
a mutation at position 309 selected from L309Q and L309T.

In certain embodiments, the IgG Fc variant comprises a single stability-enhancing mutation. In some embodiments, the IgG Fc variant comprises a single stability-enhancing mutation selected from:
a mutation at position 287 selected from A287F, A287H, A287M, A287W and A287Y;
the mutation V308I, and
a mutation at position 309 selected from L309Q and L309T.

In some embodiments, the mutation at position 287 comprised by the IgG Fc variant is A287F. In some embodiments, the mutation at position 309 comprised by the IgG Fc variant is L309Q.

In certain embodiments, the Fc variant comprises a pair of stability-enhancing mutations each of which introduces a cysteine residue allowing for formation of a new disulphide bond into the Fc region. In some embodiments, the pair of stability-enhancing mutations is selected from: L242C_I336C, V240C_I332C and V263C_V302C. In some embodiments, the pair of stability-enhancing mutations is L242C_I336C or V240C_I332C. In some embodiments, the pair of stability-enhancing mutations is L242C_I336C.

In certain embodiments, the IgG Fc variant comprises two or more stability-enhancing mutations. In some embodiments, the IgG Fc variant comprises two or more stability-enhancing mutations selected from:
a mutation at position 250 selected from T250A, T250I and T250V;
a mutation at position 287 is selected from A287F, A287H, A287M, A287W and A287Y;
the mutation V308I;
a mutation at position 309 selected from L309Q and L309T;
the mutation M428F, and
the mutations L242C and I336C.

In certain embodiments, the IgG Fc variant comprises two stability-enhancing mutations. In some embodiments, the IgG Fc variant comprises:
two stability-enhancing mutations selected from: a mutation at position 250, a mutation at position 287, a mutation at position 308, a mutation at position 309 and a mutation at position 428, where the mutation at position 250 is selected from T250A, T250I and T250V; the mutation at position 287 is selected from A287F, A287H, A287M, A287W and A287Y; the mutation at position 308 is V308I; the mutation at position 309 is selected from L309Q and L309T, and the mutation at position 428 is M428F, or
the mutations L242C and I336C.

In some embodiments, the IgG Fc variant comprises:
a mutation at position 250 selected from T250A, T250I and T250V, and a mutation at position 287 selected from A287F, A287H, A287M, A287W and A287Y;
a mutation at position 250 selected from T250A, T250I and T250V, and the mutation V308I;
a mutation at position 250 selected from T250A, T250I and T250V, and a mutation at position 309 selected from L309Q and L309T;
a mutation at position 250 selected from T250A, T250I and T250V, and the mutation M428F;
a mutation at position 287 selected from A287F, A287H, A287M, A287W and A287Y, and the mutation V308I;
a mutation at position 287 selected from A287F, A287H, A287M, A287W and A287Y, and a mutation at position 309 selected from L309Q and L309T;
a mutation at position 287 selected from A287F, A287H, A287M, A287W and A287Y, and the mutation M428F;
the mutation V308I, and a mutation at position 309 selected from L309Q and L309T; the mutation V308I, and the mutation M428F;
a mutation at position 309 selected from L309Q and L309T, and the mutation M428F, or the mutations L242C and I336C.

In some embodiments, the IgG Fc variant comprises:
a mutation at position 250 selected from T250A, T250I and T250V, and a mutation at position 287 selected from A287F, A287H, A287M, A287W and A287Y;
a mutation at position 250 selected from T250A, T250I and T250V, and a mutation at position 309 selected from L309Q and L309T;
a mutation at position 250 selected from T250A, T250I and T250V, and the mutation M428F;
a mutation at position 287 selected from A287F, A287H, A287M, A287W and A287Y, and the mutation M428F, or
the mutations L242C and I336C.

In some embodiments, the mutation at position 250 comprised by the IgG Fc variant is T250V. In some embodiments, the mutation at position 287 comprised by the IgG Fc variant is A287F. In some embodiments, the mutation at position 309 comprised by the IgG Fc variant is L309Q.

In some embodiments, the IgG Fc variant comprises the stability-enhancing mutations T250V/A287F, T250V/V308I, T250V/L309Q, T250V/M428F, A287F/V308I, A287F/L309Q, A287F/M428F, V308I/L309Q, V308I/M428F, L309Q/M428F or L242C_I336C.

In some embodiments, the IgG Fc variant comprises the stability-enhancing mutations T250V/A287F, T250V/L309Q, T250V/M428F, A287F/M428F or L242C_I336C.

In some embodiments, the IgG Fc variant comprises the stability-enhancing mutations T250V/A287F, T250V/L309Q, T250V/M428F or A287F/M428F.

In some embodiments, the stability-enhancing mutations comprised by the Fc variant are selected from: T250V, A287F, V308I, L309Q, M428F, L242C_I336C, A287F/M428F, T250V/A287F, T250V/L309Q, T250V/M428F and L242C_I336C/V308I.

In some embodiments, the stability-enhancing mutations comprised by the Fc variant are selected from: A287F, V308I, L309Q, L242C_I336C, A287F/M428F, T250V/A287F, T250V/L309Q, T250V/M428F and L242C_I336C/V308I.

In certain embodiments, the IgG Fc variant comprises three or more stability-enhancing mutations. In some embodiments, the IgG Fc variant comprises three or more stability-enhancing mutations selected from:
a mutation at position 250 selected from T250A, T250I and T250V; a mutation at position 287 selected from A287F, A287H, A287M, A287W and A287Y; the mutation V308I; a mutation at position 309 selected from L309Q and L309T; the mutation M428F, and the mutations L242C and I336C.

In some embodiments, the IgG Fc variant comprises three stability-enhancing mutations. In some embodiments, the IgG Fc variant comprises three stability-enhancing mutations selected from:
a mutation at position 250 selected from T250A, T250I and T250V; a mutation at position 287 selected from A287F, A287H, A287M, A287W and A287Y; the mutation V308I; a mutation at position 309 selected from L309Q and L309T; the mutation M428F, and the mutations L242C and I336C.

In some embodiments, the IgG Fc variant comprises the mutations L242C and 1336C and a mutation selected from: a mutation at position 250 selected from T250A, T250I and T250V; a mutation at position 287 selected from A287F, A287H, A287M, A287W and A287Y; the mutation V308I; a mutation at position 309 selected from L309Q and L309T, and the mutation M428F.

In certain embodiments, the IgG Fc variant comprises between one and three stability-enhancing mutations. In some embodiments, the IgG Fc variant comprises:
one or more mutation selected from: a mutation at position 287 selected from A287F, A287H, A287M, A287W and A287Y; the mutation V308I, and a mutation at position 309 selected from L309Q and L309T, or
two or more mutations selected from: a mutation at position 250 selected from T250A, T250I and T250V; a mutation at position 287 selected from A287F, A287H, A287M, A287W and A287Y; the mutation V308I; a mutation at position 309 selected from L309Q and L309T; the mutation M428F, and the mutations L242C and 1336C, or
three or more mutations comprising: the mutations L242C and I336C, and a mutation selected from: a mutation at position 250 selected from T250A, T250I and T250V; a mutation at position 287 selected from A287F, A287H, A287M, A287W and A287Y; the mutation V308I; a mutation at position 309 selected from L309Q and L309T, and the mutation M428F.

Certain stability-enhancing mutations are known in the art. For example, introduction of additional disulphide bonds by including the mutations L242C_K334C, L240C_K334C, A287C_L306C, V259C_L306C, R292C_V302C or V323C_I332C in the Fc region has been shown to increase stability (Gong et al., 2009, J Biol Chem, 284(21):14203-14210; Jacobsen et al., 2017, J Boil Chem, 292(5):1865-1875). Other stability-enhancing mutations are described in U.S. Patent Application Publication No. 2015/0210763. Certain embodiments of the present disclosure contemplate Fc variants comprising a combination of one or more of the stability-enhancing mutations disclosed herein with one or more mutations previously shown to increase the stability of the Fc region.

### Methods

Certain embodiments of the present disclosure relate to methods of stabilizing an Fc region (the parental Fc) by introducing one or more stability-enhancing mutations as described herein into the parental Fc to provide an Fc variant.

Some embodiments of the present disclosure relate to methods of increasing the CH2 domain melting temperature (Tm) of an Fc region (the parental Fc) by introducing one or more stability-enhancing mutations as described herein into the parental Fc to provide an Fc variant having an increased CH2 domain Tm of at least 0.5° C. as compared to the parental Fc.

Some embodiments of the present disclosure relate to methods of increasing the CH2 domain Tm of a parental Fc, the method comprising introducing one or more stability-enhancing mutations as described herein into the Fc to provide an Fc variant, where the Fc variant has a CH2 domain Tm at least 0.5° C. higher than the CH2 domain Tm of the parental Fc.

The parental Fc may be a wild-type Fc or it may itself be a variant Fc that already includes one or more amino acid mutations, for example, to improve a function of the Fc region. In some embodiments, the parental Fc may comprise one or more amino acid mutations that improve a function of the Fc region but also decrease the CH2 domain Tm.

Some embodiments of the present disclosure relate to methods of increasing the CH2 domain Tm of a parental Fc having a lower CH2 domain Tm than the corresponding wild-type Fc, the method comprising introducing one or more stability-enhancing mutations as described herein into the Fc to provide an Fc variant, where the Fc variant has a CH2 domain Tm at least 0.5° C. higher than the CH2 domain Tm of the parental Fc.

In some embodiments, the methods provide an Fc variant having a CH2 domain Tm at least 1.0° C., at least 2.0° C., or at least 3.0° C., higher than the CH2 domain Tm of the parental Fc.

In some embodiments, the methods provide an Fc variant having a CH2 domain Tm between about 0.5° C. and about 6.5° C. higher than the CH2 domain Tm of the parental Fc. In some embodiments, the CH2 domain Tm of the Fc variant is between about 0.5° C. and about 9.0° C., between about 1.0° C. and about 9.0° C., between about 2.0° C. and about 9.0° C., or between about 3.0° C. and about 9.0° C., higher than the CH2 domain Tm of the parental Fc. In some embodiments, the CH2 domain Tm of the Fc variant is between about 2.0° C. and about 10.5° C., or between about 3.0° C. and about 10.5° C., higher than the CH2 domain Tm of the parental Fc.

In certain embodiments, the methods further comprise measuring the CH2 domain Tm of the Fc variant. In some embodiments, the methods further comprise measuring the CH2 domain Tm of the Fc variant by DSC or DSF.

In certain embodiments, the methods comprise introducing between one and five stability-enhancing mutations as described herein into the parental Fc. In some embodiments, the methods comprise introducing between one and four stability-enhancing mutations as described herein into the parental Fc. In some embodiments, the methods comprise introducing between one and three stability-enhancing mutations as described herein into the parental Fc. In some embodiments, the methods comprise introducing 1, 2 or 3 stability-enhancing mutations into the parental Fc.

In some embodiments, the methods comprise introducing into the parental Fc one or more stability-enhancing mutations selected from:
a mutation at position 250, where the mutation is a substitution of the amino acid at position 250 with Ala, Ile or Val;
a mutation at position 287, where the mutation is a substitution of the amino acid at position 287 with Phe, His, Met, Trp or Tyr;
a mutation at position 308, where the mutation is a substitution of the amino acid at position 308 with Ile;
a mutation at position 309, where the mutation is a substitution of the amino acid at position 309 with Gln or Thr;
a mutation at position 428, where the mutation is a substitution the amino acid at position 428 with Phe, and
a mutation at position 242 and a mutation at position 336, where both mutations are substitutions with Cys.

In some embodiments, the methods comprise introducing into the parental Fc a single stability-enhancing amino acid mutation selected from: a mutation at position 287 which is a substitution with Phe, His, Met, Trp or Tyr; a mutation at position 308 which is a substitution with Ile; a mutation at position 309 which is a substitution with Gln or Thr.

In some embodiments, the methods comprise introducing into the parental Fc two or more stability-enhancing amino acid mutations selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr; a mutation at position 428 which is a substitution with Phe, and a mutation at position 242 and a mutation at position 336 which are both substitutions with Cys.

In some embodiments, the methods comprise introducing into the parental Fc three or more stability-enhancing amino acid mutations comprising: a mutation at position 242 and a mutation at position 336 which are both substitutions with Cys, and a mutation selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr, and a mutation at position 428 which is a substitution with Phe.

Certain embodiments relate to a method of increasing the CH2 domain Tm of an Fc region (the parental Fc) comprising introducing into the parental Fc one to three stability-enhancing amino acid mutations to provide an Fc variant having an increased CH2 domain Tm as compared to the parental Fc region, where the one to three stability-enhancing mutations comprise:
(a) one or more mutation selected from: a mutation at position 287 which is a substitution with Phe, His, Met, Trp or Tyr; a mutation at position 308 which is a substitution with Ile; a mutation at position 309 which is a substitution with Gln or Thr, or
(b) two or more mutations selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr; a mutation at position 428 which is a substitution with Phe, and a mutation at position 242 and a mutation at position 336 which are both substitutions with Cys, or
(c) three or more mutations comprising: a mutation at position 242 and a mutation at position 336 which are both substitutions with Cys, and a mutation selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr, and a mutation at position 428 which is a substitution with Phe.

In some embodiments, the methods comprise introducing the stability-enhancing mutations 250V/287F, 250V/308I, 250V/309Q, 250V/428F, 287F/308I, 287F/309Q, 287F/428F, 308I/309Q, 308I/428F, 309Q/428F or 242C_336C into the parental Fc. In some embodiments, the methods comprise introducing the stability-enhancing mutations 250V/287F, 250V/309Q, 250V/428F, 287F/428F or 242C_336C into the parental Fc. In some embodiments, the methods comprise introducing the stability-enhancing mutations 250V/287F, 250V/309Q, 250V/428F or 287F/428F into the parental Fc.

In certain embodiments, the methods comprise introducing stability-enhancing mutations selected from: 250V, 287F, 308I, 309Q, 428F, 242C_336C, 287F/428F, 250V/287F, 250V/309Q, 250V/428F and 242C_336C/308I, into the parental Fc. In some embodiments, the methods comprise introducing stability-enhancing mutations selected from: 287F, 308I, 309Q, 242C_336C, 287F/428F, 250V/287F, 250V/309Q, 250V/428F and 242C_336C/308I, into the parental Fc.

In certain embodiments, the parental Fc is an IgG, IgA, IgD, IgE or IgM Fc, for example a human IgG, IgA, IgD, IgE or IgM Fc. In some embodiments, the parental Fc is an IgG or IgA Fc, for example a human IgG or IgA Fc. In some embodiments, the parental Fc is an IgG Fc, for example a human IgG Fc.

In certain embodiments, the parental Fc is an IgG1, IgG2, IgG3 or IgG4 Fc, for example a human IgG1, IgG2, IgG3 or IgG4 Fc. In some embodiments, the parental Fc is an IgG1 Fc, for example a human IgG1 Fc.

In certain embodiments, the methods provide an Fc variant having an increased CH2 domain Tm of at least 0.5° C. as compared to the parental Fc and show decreased aggregation of the Fc region as compared to the parental Fc. In some embodiments, the Fc variant produced by the methods has an increased CH2 domain Tm of at least 0.5° C. as compared to the parental Fc and shows decreased aggregation of the Fc region at low pH as compared to the parental Fc. In some embodiments, the Fc variant produced by the methods has an increased CH2 domain Tm of at least 0.5° C. as compared to the parental Fc and shows decreased aggregation under mildly acidic conditions as compared to the parental Fc.

### Assays

The Fc variants of the present disclosure have increased stability as compared to the parental Fc. This increased stability may result in increased thermostability of the CH2 domain, decreased aggregation, increased serum half-life, increased manufacturability, or a combination thereof.

In certain embodiments, the Fc variant has increased thermostability over the parental Fc as determined by CH2 domain melting temperature (Tm). The CH2 domain Tm of the Fc variant and parental Fc may be measured, for example, by circular dichroism (CD), differential scanning calorimetry (DSC) or differential scanning fluorimetry (DSF) using standard techniques. In certain embodiments, the Fc variants have increased stability over the parental Fc as determined by CH2 domain Tm, where the CH2 domain Tm is measured by DSC or DSF.

In certain embodiments, the stability-enhancing mutations result in an increase in CH2 domain Tm of the Fc variant over the parental Fc of at least 0.5° C. when introduced into the Fc as a single mutation. In some embodiments, the stability-enhancing mutations result in an increase in CH2 domain Tm of the Fc variant over the parental Fc of at least 1.0° C., at least 1.5° C., at least 2.0° C., at least 2.5° C. or at least 3.0° C., when introduced into the Fc as a single mutation. In some embodiments, the stability-enhancing mutations result in an increase in CH2 domain Tm of the Fc variant over the parental Fc of between about 0.5° C. and about 6.5° C. when introduced into the Fc as a single mutation.

In certain embodiments, the stability-enhancing mutations result in an increase in CH2 domain Tm of the Fc variant over the parental Fc of at least 0.5° C. when introduced into the Fc as combinations of two or more mutations. In some embodiments, the stability-enhancing mutations result in an increase in CH2 domain Tm of the Fc variant over the parental Fc of at least 1.0° C., at least 1.5° C., at least 2.0° C., at least 2.5° C. or at least 3.0° C., when introduced into the Fc as combinations of two or more mutations. In some embodiments, the stability-enhancing mutations result in an increase in CH2 domain Tm of the Fc variant over the parental Fc of at least 5.0° C., at least 5.5° C., at least 6.0° C., at least 6.5° C. or at least 7.0° C., when introduced into the Fc as combinations of two or more mutations. In some embodiments, the stability-enhancing mutations result in an increase in CH2 domain Tm of the Fc variant over the parental Fc of between about 0.5° C. and about 9.0° C. when introduced into the Fc as combinations of two or more mutations. In some embodiments, the stability-enhancing mutations result in an increase in CH2 domain Tm of the Fc variant over the parental Fc of between about 1.0° C. and about 9.0° C., between about 2.0° C. and about 9.0° C., or between about 3.0° C. and about 9.0° C. when introduced into the Fc as combinations of two or more mutations. In some embodiments, the stability-enhancing mutations result in an increase in CH2 domain Tm of the Fc variant over the parental Fc of between about 2.0° C. and about 10.5° C., or between about 3.0° C. and about 10.5° C., when introduced into the Fc as combinations of two or more mutations.

In certain embodiments, the increased stability of the Fc variants results in decreased aggregation and/or increased serum half-life of the Fc variant as compared to the parental Fc. Aggregation and serum half-life may be measured by various standard techniques known in the art. For example, aggregation of the Fc variant and parental Fc may be assessed by size-exclusion chromatography (SEC) or dynamic light scattering (DLS). Serum half-life of the Fc variant and parental Fc may be assessed, for example, by pharmacokinetic studies in model animals.

In certain embodiments, the Fc variant has increased thermostability over the parental Fc as determined by CH2 domain melting temperature (Tm) and also shows decreased aggregation. In some embodiments, the Fc variant has increased thermostability (Tm) over the parental Fc and also shows decreased aggregation at low pH. In some embodiments, the Fc variant has increased thermostability (Tm) over the parental Fc and also shows decreased aggregation under mildly acidic conditions.

Other assays may optionally be conducted using standard techniques in order to further characterize the Fc variants. For example, the Fc variants may be assessed for purity, FcR binding, FcRn binding, aggregation and/or C1q binding. Purity and aggregation may be assessed, for example, by liquid chromatography-mass spectrometry (LC-MS) and size-exclusion chromatography (SEC), respectively. FcR and FcRn binding may be measured, for example, by surface plasmon resonance (SPR), SPR imaging (SPRi), bio-layer interferometry (BLI), ELISA, Kinetic Exclusion Assay (KinExA®) or Meso Scale Discovery™ (MSD™)-based methods (see, for example, Current Protocols in Immunology: Ligand-Receptor Interactions in the Immune System, Eds. J. Coligan et al., 2018 & updates, Wiley Inc., Hoboken, NJ; and Yang et al., 2016, Analytical Biochem, 508:78-96). C1q binding may be assessed, for example, by ELISA or SPR.

In certain embodiments, the Fc variants are IgG Fc variants and may be assessed for FcγR binding and/or FcRn binding. Typically, binding affinity is expressed in terms of the dissociation constant (K_{D}) for binding of the Fc variant to the FcγR or FcRn. In some embodiments in which the Fc variants are IgG Fc variants, the Fc variants retain substantially the same binding to each of the Fcγ receptors as the parental Fc. In some embodiments in which the Fc variants are IgG Fc variants, the Fc variants retain substantially the same binding to FcRn as the parental Fc. “Substantially the same binding” in this context means a change of 3-fold or less in K_{D} as compared to the parental Fc.

### Polypeptides

Certain embodiments of the present disclosure relate to polypeptides comprising an Fc variant as described herein. Typically, the polypeptides comprise one or more additional proteinaceous moieties fused to the Fc variant or covalently attached to the Fc variant, for example, by means of a linker. For example, the polypeptide may be an Fc fusion protein or an antibody or antibody fragment. Examples of proteinaceous moieties that may be fused or attached to the Fc variant include, but are not limited to, antigen-binding domains, ligands, receptors, receptor fragments, cytokines and antigens.

When the polypeptides comprise more than one additional proteinaceous moiety, the moieties may be the same or they may be different. The one or more additional proteinaceous moieties may be fused at the N-terminus, the C-terminus or both the N-terminus and the C-terminus of one or both of the Fc polypeptides. In some embodiments, the polypeptides comprise one or more additional proteinaceous moieties fused to the N-terminus of one or both of the Fc polypeptides. In some embodiments, the polypeptides comprise one additional proteinaceous moiety fused to the N-terminus of one of the Fc polypeptides. In some embodiments, the polypeptides comprise two additional proteinaceous moieties, one moiety fused to the N-terminus of the first Fc polypeptide and the other moiety fused to the N-terminus of the second Fc polypeptide. In some embodiments, two additional proteinaceous moieties comprised by the polypeptides may be linked in tandem.

In some embodiments, the polypeptides comprise an Fc variant fused to one or more proteinaceous moieties that are antigen-binding domains. In some embodiments, the polypeptides comprise an Fc variant and one or more antigen-binding domains. In some embodiments, the polypeptides comprise an Fc variant and two or more antigen-binding domains, for example, 2, 3, 4, 5, 6, 7 or 8 antigen-binding domains. When the polypeptide comprises an Fc variant and two or more antigen-binding domains, the antigen-binding domains may bind the same antigen or they may bind different antigens.

In some embodiments, the polypeptides comprise an Fc variant fused to one or more proteinaceous moieties that are antigen-binding domains and to one or more other proteinaceous moieties. In some embodiments, the polypeptides comprise an Fc variant fused to an antigen-binding domain and to one or more other proteinaceous moieties. Examples of other proteinaceous moieties in this context include, but are not limited to, receptors, receptor fragments (such as extracellular portions), ligands and cytokines.

In some embodiments, the polypeptide may be an antibody or an antibody fragment in which at least one of the one or more proteinaceous moieties is an antigen-binding domain. For example, the antigen-binding domain may be a Fab fragment, Fv fragment, single-chain Fv fragment (scFv) or single domain antibody (sdAb). In some embodiments, the polypeptide may be a monospecific antibody. In some embodiments, the polypeptide may be a monospecific antibody comprising one antigen-binding domain. In some embodiments, the polypeptide may be a monospecific antibody comprising two antigen-binding domains. In some embodiments, the polypeptide may be a monospecific antibody comprising more than two antigen-binding domains. In some embodiments, the polypeptide may be a bispecific or multispecific antibody comprising an Fc variant and two or more antigen-binding domains, in which the two or more antigen-binding domains bind to different antigens.

In some embodiments, the polypeptide may be a therapeutic or diagnostic antibody or antibody fragment in which at least one of the one or more proteinaceous moieties is an antigen-binding domain.

In some embodiments, the polypeptides comprise an Fc variant and one or more antigen-binding domains that bind to tumour-associated antigens or tumour-specific antigens.

### Preparation of Fc Variants

The Fc variants described herein and polypeptides comprising an Fc variant as described herein may be prepared using standard recombinant methods. Recombinant production of the Fc variants and polypeptides generally involves synthesizing one or more polynucleotides encoding the Fc variant or polypeptide, cloning the one or more polynucleotides into an appropriate vector or vectors, and introducing the vector(s) into a suitable host cell for expression of the Fc variant or polypeptide. Recombinant production of proteins is well-known in the art and may be achieved using standard techniques as described, for example, in Sambrook et al., Molecular Cloning: A Laboratory Manual, 3rd ed., Cold Spring Harbor Laboratory Press, Cold Spring Harbor, NY (2001); Ausubel et al., Current Protocols in Molecular Biology, (1987 & updates), John Wiley & Sons, New York, NY; and Harlow and Lane, Antibodies: A Laboratory Manual, Cold Spring Harbor Laboratory Press, Cold Spring Harbor, NY (1990).

Certain embodiments of the present disclosure thus relate to an isolated polynucleotide or set of polynucleotides encoding an Fc variant as described herein or encoding a polypeptide comprising an Fc variant as described herein. A polynucleotide in this context may encode all or part of an Fc variant or polypeptide.

The terms “nucleic acid,” “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, isolated DNA, isolated RNA, nucleic acid probes, and primers.

A polynucleotide that “encodes” a given polypeptide is a polynucleotide that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

The one or more polynucleotides encoding the Fc variant or polypeptide may be inserted into a suitable expression vector or vectors, either directly or after one or more subcloning steps, using standard ligation techniques. Examples of suitable vectors include, but are not limited to, plasmids, phagemids, cosmids, bacteriophage, baculoviruses, retroviruses or DNA viruses. The vector is typically selected to be functional in the particular host cell that will be employed, i.e. the vector is compatible with the host cell machinery, permitting amplification and/or expression of the polynucleotide(s). Selection of appropriate vector and host cell combinations in this regard is well within the ordinary skills of a worker in the art.

Certain embodiments of the present disclosure thus relate to vectors (such as expression vectors) comprising one or more polynucleotides encoding an Fc variant or polypeptide comprising an Fc variant. The polynucleotide(s) may be comprised by a single vector or by more than one vector. In some embodiments, the polynucleotides are comprised by a multicistronic vector.

Typically, expression vectors will contain one or more regulatory elements for plasmid maintenance and for cloning and expression of exogenous polynucleotide sequences. Examples of such regulatory elements include promoters, enhancer sequences, origins of replication, transcriptional termination sequences, donor and acceptor splice sites, leader sequences for polypeptide secretion, ribosome binding sites, polyadenylation sequences, polylinker regions for inserting the polynucleotide encoding the polypeptide to be expressed, and selectable markers.

Regulatory elements may be homologous (i.e. from the same species and/or strain as the host cell), heterologous (i.e. from a species other than the host cell species or strain), hybrid (i.e. a combination of regulatory elements from more than one source) or synthetic. As such, the source of a regulatory element may be any prokaryotic or eukaryotic organism provided that the sequence is functional in, and can be activated by, the machinery of the host cell being employed.

Optionally, the vector may also contain a “tag”-encoding sequence. A tag-encoding sequence is a nucleic acid sequence located at the 5′ or 3′ end of the coding sequence that encodes a heterologous peptide sequence, such as a polyHis (for example, 6xHis), FLAG®, HA (hemaglutinin influenza virus), myc, metal-affinity, avidin/streptavidin, glutathione-S-transferase (GST) or biotin tag. This tag typically remains fused to the expressed polypeptide and can serve as a means for affinity purification or detection of the polypeptide. Optionally, the tag can subsequently be removed from the purified polypeptide by various means such as using certain peptidases for cleavage.

Various expression vectors are readily available from commercial sources. Alternatively, when a commercial vector containing all the desired regulatory elements is not available, an expression vector may be constructed using a commercially available vector as a starting vector. Where one or more of the desired regulatory elements are not already present in the vector, they may be individually obtained and ligated into the vector. Methods and sources for obtaining various regulatory elements are well known to one skilled in the art.

Following construction of the expression vector(s) including the polynucleotide(s) encoding the Fc variant or polypeptide, the vector(s) may be inserted into a suitable host cell for amplification and/or protein expression. The transformation of an expression vector into a selected host cell may be accomplished by well-known methods including transfection, infection, calcium phosphate co-precipitation, electroporation, microinjection, lipofection, DEAE-dextran mediated transfection, and other known techniques. The method selected will in part be a function of the type of host cell to be used. These methods and other suitable methods are well known to the skilled person (see, for example, Sambrook, et al., ibid.).

A host cell, when cultured under appropriate conditions, expresses the polypeptide encoded by the vector and the polypeptide can subsequently be collected from the culture medium (if the host cell secretes the polypeptide) or directly from the host cell producing it (if the polypeptide is not secreted). The host cell may be prokaryotic (for example, a bacterial cell) or eukaryotic (for example, a yeast, fungi, plant or mammalian cell). The selection of an appropriate host cell can be readily made by the skilled person taking into account various factors, such as desired expression levels, polypeptide modifications that are desirable or necessary for activity (such as glycosylation or phosphorylation) and ease of folding into a biologically active molecule.

Certain embodiments of the present disclosure thus relate to host cells comprising polynucleotide(s) encoding the Fc variant or the polypeptide comprising the Fc variant, or one or more vectors comprising the polynucleotide(s). In certain embodiments, the host cell is a eukaryotic cell.

For example, eukaryotic microbes such as filamentous fungi or yeast may be employed as host cells, including fungi and yeast strains whose glycosylation pathways have been “humanized” (see, for example, Gerngross, (2004), Nat. Biotech., 22:1409-1414, and Li et al., (2006), Nat. Biotech., 24:210-215). Plant cells may also be utilized as host cells (see, for example, U.S. Pat. Nos. 5,959,177; 6,040,498; 6,420,548; 7,125,978 and 6,417,429, describing PLANTIBODIES™ technology).

In some embodiments, the eukaryotic host cell is a mammalian cell. Various mammalian cell lines may be used as host cells. Examples of useful mammalian host cell lines include, but are not limited to, monkey kidney CV1 line transformed by SV40 (COS-7), human embryonic kidney line 293 (HEK293 cells as described, for example, in Graham, et al., (1977), J. Gen Virol., 36:59), baby hamster kidney cells (BHK), mouse sertoli cells (TM4 cells as described, for example, in Mather, (1980), Biol. Reprod., 23:243-251), monkey kidney cells (CV1), African green monkey kidney cells (VERO-76), human cervical carcinoma cells (HeLa), canine kidney cells (MDCK), buffalo rat liver cells (BRL 3A), human lung cells (W138), human liver cells (Hep G2), mouse mammary tumour cells (MMT 060562), TRI cells (as described, for example, in Mather, et al., 1982, Annals N.Y. Acad. Sci., 383:44-68), MRC 5 cells, FS4 cells, Chinese hamster ovary (CHO) cells (including DHFR CHO cells as described in Urlaub, et al., 1980, Proc. Natl. Acad. Sci. USA, 77:4216) and myeloma cell lines (such as YO, NSO and Sp2/0). See also, Yazaki and Wu, 2003, Methods in Molecular Biology, Vol. 248, pp. 255-268 (B. K. C. Lo, ed., Humana Press, Totowa, N.J.).

Certain embodiments of the present disclosure relate to methods of preparing an Fe variant as described herein or a polypeptide comprising an Fc variant as described herein, comprising transfecting a host cell with one or more polynucleotides encoding the Fc variant or polypeptide, for example in the form of one or more vectors comprising the polynucleotide(s), and culturing the host cell under conditions suitable for expression of the encoded Fc variant or polypeptide.

Typically, the Fc variant or polypeptide is isolated from the host cell after expression and may optionally be purified. Methods for isolating and purifying expressed proteins are well-known in the art. Standard purification methods include, for example, chromatographic techniques, such ion exchange, hydrophobic interaction, affinity, sizing, gel filtration or reverse-phase, which may be carried out at atmospheric pressure or at medium or high pressure using systems such as FPLC, MPLC and HPLC. Other purification methods include electrophoretic, immunological, precipitation, dialysis and chromatofocusing techniques. Ultrafiltration and diafiltration techniques, in conjunction with protein concentration, may also be useful.

A variety of natural proteins are known in the art to bind Fc regions of antibodies, and these proteins can therefore be used in the purification of Fc-containing proteins. For example, the bacterial proteins A and G bind to the Fc region. Purification can often be enabled by a particular fusion partner or affinity tag as described above. For example, antibodies may be purified using glutathione resin if a GST fusion is employed, Ni⁺² affinity chromatography if a His-tag is employed, or immobilized anti-flag antibody if a FLAG-tag is used. Examples of useful purification techniques are described in Harlow and Lane, Antibodies: A Laboratory Manual, Cold Spring Harbor Laboratory Press, Cold Spring Harbor, NY (1990), and Protein Purification: Principles and Practice, 3^{rd} Ed., Scopes, Springer-Verlag, NY (1994).

### Pharmaceutical Compositions

Certain embodiments of the present disclosure relate to the therapeutic use of the Fc variants or polypeptides comprising an Fc variant. For therapeutic use, the Fc variants and polypeptides may be provided in the form of compositions which comprise the Fc variant or polypeptide and a pharmaceutically acceptable carrier or diluent. The compositions may be prepared by known procedures using well-known and readily available ingredients and may be formulated for administration to a subject by, for example, oral (including, for example, buccal or sublingual), topical, parenteral, rectal or vaginal routes, or by inhalation or spray. The term “parenteral” as used herein includes injection or infusion by subcutaneous, intradermal, intra-articular, intravenous, intramuscular, intravascular, intrasternal or intrathecal routes.

The composition will typically be formulated in a format suitable for administration to a subject by the chosen route, for example, as a syrup, elixir, tablet, troche, lozenge, hard or soft capsule, pill, suppository, oily or aqueous suspension, dispersible powder or granule, emulsion, injectable or solution. Compositions may be provided as unit dosage formulations.

Pharmaceutically acceptable carriers are generally non-toxic to recipients at the dosages and concentrations employed. Examples of such carriers include, but are not limited to, buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 amino acids) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, the compositions may be in the form of a sterile injectable aqueous or oleaginous solution or suspension. Such suspensions may be formulated using suitable dispersing or wetting agents and/or suspending agents that are known in the art. The sterile injectable solution or suspension may comprise the Fc variant or polypeptide in a non-toxic parentally acceptable diluent or solvent. Acceptable diluents and solvents that may be employed include, for example, 1,3-butanediol, water, Ringer's solution or isotonic sodium chloride solution. In addition, sterile, fixed oils may be employed as a solvent or suspending medium. For this purpose, various bland fixed oils may be employed, including synthetic mono- or diglycerides. In addition, fatty acids such as oleic acid find use in the preparation of injectables. Adjuvants such as local anaesthetics, preservatives and/or buffering agents as known in the art may also be included in the injectable solution or suspension.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Methods of Use

Certain embodiments of the present disclosure relate to the use of the Fc variants or polypeptides comprising the Fc variants as therapeutics, diagnostics or research tools. Some embodiments relate to the therapeutic use of the Fc variants and polypeptides comprising the Fc variants.

Polypeptides comprising an Fc variant as described herein and one or more antigen-binding domains, for example, antibodies or antibody fragments, are especially useful as diagnostics and therapeutics. Some embodiments thus relate to methods of using a polypeptide comprising an Fc variant and one or more antigen-binding domains in the diagnosis of a disease or disorder in a subject. Some embodiments relate to methods of using a polypeptide comprising an Fc variant and one or more antigen-binding domains in the treatment of a disease or disorder in a subject in need thereof.

The disease or disorder to be diagnosed or treated will be dependent on the antigen or antigens being targeted by the antigen-binding domains. Examples of diseases and disorders that may be diagnosed or treated include, but are not limited to, inflammatory diseases and disorders, autoimmune diseases and disorders, and proliferative diseases and disorders, such as various cancers.

The following Examples are provided for illustrative purposes and are not intended to limit the scope of the claimed invention in any way.

### EXAMPLES

### General Methods

### Preparation of Variants

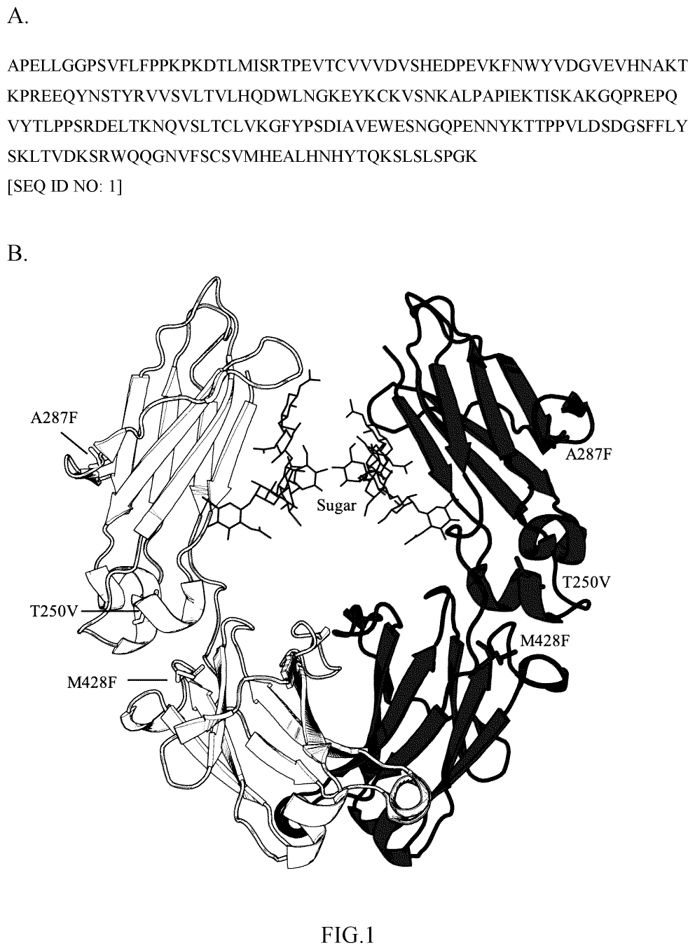

Variants and controls were prepared by site-directed mutagenesis and/or restriction/ligation using standard methods. The final DNA was sub-cloned into the vector pTT5 (see U.S. Pat. No. 9,353,382). All scaffolds used for preparation of the variants were based on an IgG1 Fc. The sequence of the IgG1 Fc region is provided in FIG. 1A. In certain clones, the C-terminal lysine residue was omitted from the Fc sequence.

The following scaffolds were used:

Scaffold 1: Full-size antibody (FSA) based on trastuzumab with homodimeric IgG1 Fc, SEQ ID NO:1.

Scaffold 2: One-armed antibody (OAA) scaffold comprising a trastuzumab Fab and a heterodimeric IgG1 Fc comprising the following mutations:
Chain A: T350V_L351Y_F405A_Y407V
Chain B: T350V_T366L_K392L_T394W

Scaffold 3: Full-size antibody (FSA) based on trastuzumab comprising the same heterodimeric Fc as for Scaffold 2.

Scaffold 4: Full-size antibody (FSA) based on the 4G7 anti-CD19 antibody (Meeker, et al., 1984, Hybridoma, 3:305-320; U.S. Pat. No. 8,524,867) comprising the same heterodimeric Fc as for Scaffold 2.

Scaffold 5: Full-size antibody (FSA) based on the CP-870,893 anti-CD40 antibody (Gladue, et al., 2011, Cancer Immunol Immunother, 60:1009-1017) comprising the same heterodimeric Fc as for Scaffold 2. Variable domain sequence was obtained from International Patent Application Publication No. WO 2013/132044.

Scaffold 6: Full-size antibody (FSA) based on trastuzumab comprising the N297A mutation (Leabman, et al, 2013, mAb, 5(6):896-903) which results in an aglycosylated Fc and abrogated binding to all FcγRs.

Scaffold 7: Full-size antibody (FSA) based on trastuzumab comprising the S239D and 1332E mutations (Lazar, et al, 2006, PNAS, 103:4005-4010) which result in increased FcγRIIIa binding.

Scaffold 8: One-armed antibody (OAA) scaffold comprising a trastuzumab Fab and a heterodimeric IgG1 Fc comprising the following mutations which result in increased FcγRIIb selectivity:
Chain A: CH2: G236N G237A CH3: T350V_L351Y_F405A_Y407V
Chain B: CH2: G236D_G237F_S239D_S267V_H268D_“Template 1” CH3: T350V_T366L_K392L_T394W

“Template 1” indicates a replacement of the amino acid residues at positions 325-331 with the following sequence: STWFDGGYAT [SEQ ID NO:2].

Scaffold 9: One-armed antibody (OAA) scaffold comprising a trastuzumab Fab and a heterodimeric IgG1 Fc comprising the following mutations which result in increased FcγRIIb selectivity:
Chain A: CH2: L234F_G236N_H268Q A327G_A330K_P331S CH3: T350V_L351Y_F405A_Y407V
Chain B: CH2: G236D_S239D_V266L_S267A_H268D CH3: T350V_T366L_K392L_T394W

### Expression—Protocol 1

Expression was carried out in 200 mL CHO 3E7 cells. CHO cells were transfected in exponential growth phase (1.5 to 2 million cells/mL) with aqueous 1 mg/mL 25 kDa polyethylenimine (PEI^{pro}, Polyplus Transfection SA, Illkirch, France) at a PEI:DNA ratio of 2.5:1 (Delafosse, et al., 2016, J. Biotechnol., 227:103-111). In order to determine the optimal concentration range for forming heterodimers, the DNA was transfected in optimal DNA ratios of the heavy chain A (HC-A), light chain (LC), and heavy chain B (HC-B) that allow for heterodimer formation (e.g., HC-A/HC-B/LC ratios=30:30:40%). When expressing homodimers, ratios of 50:50% were used for HC:LC. Transfected cells were harvested after 5-6 days with the culture medium collected after centrifugation at 4000 rpm and clarified using a 0.45 m filter.

The clarified culture medium was loaded onto a MabSelect™ SuRe™ (GE Healthcare, Baie-d'Urfé, QC, Canada) Protein-A column and washed with 10 column volumes of PBS buffer at pH 7.2. The antibody was eluted with 10 column volumes of citrate buffer at pH 3.6 with the pooled fractions containing the antibody neutralized with TRIS at pH 11. Samples were then buffer exchanged in PBS pH 7.4 and stored at −80° C.

### Expression—Protocol 2

Expression was performed using HEK 293-6E cells (NRC, Canada) on either small-scale (1 mL) or large-scale (30 mL or greater).

For 1 mL-scale expressions, HEK 293-6E cells were transfected in exponential growth phase (1.5 to 2.0 million cells/mL) with 1 μg DNA/mL cells using DNA pre-complexed with the cationic lipid 293Fectin™ (Life Technologies, Paisley, U.K.). Heavy chain and light chain DNA were mixed at a ratio of 47.5:52.5% and DNA was complexed with 293Fectin™ at final concentrations of 11.7 μg/mL DNA, 1.65% (v/v) 293Fectin™ then incubated at ambient temperature for 30 min before addition to cells. In order to achieve optimal heterodimer formation, the ratio of the HC-A and HC-B DNA of transfection mixes was either 50:50%, or a small variation thereof. Cells were cultured for 5-6 days in a humidified shaking incubator at 37° C. and 5% carbon dioxide in a 96-well deep well plate sealed with a gas-permeable seal. Culture medium was then collected after centrifugation at 1600×g.

For large-scale expressions, HEK 293-6E cells were transfected in exponential growth phase (1.5 to 2.0 million cells/mL) with 1 μg DNA/mL cells using DNA pre-complexed with a Gemini cationic lipid (Camilleri et al., 2000, Chem. Commun., 1253-1254). Heavy chain and light chain DNA were mixed at a ratio of 50:50% and DNA was complexed with Gemini at final concentration of 10 μg/mL DNA, 40 μg/mL Gemini then incubated at ambient temperature for 15-30 min before addition to cells. HC-A and HC-B DNA ratios of transfection mixes was as described above. Cells were cultured for up to 10 days in a humidified shaking incubator at 37° C. and 5% carbon dioxide in an appropriately sized Erlenmeyer flask or BioReactor tube. Culture medium was then collected after centrifugation at 2750×g and clarified using a 0.22 μm filter.

The clarified culture medium was loaded onto a MabSelect™ SuRe™ (GE Healthcare, Little Chalfont, U.K.) protein A column, washed with 3-10 column volumes of Tris-Acetate buffer at pH7.5, then eluted with 2-5 column volumes of acetic acid at pH 2.6 with the elution fraction neutralized with TRIS. Further purification by size exclusion chromatography (Superdex™ 200 column (GE Healthcare, Little Chalfont, U.K.) with PBS running buffer) and/or cationic exchange (ReSource™ S column (GE Healthcare, Little Chalfont, U.K.)) was utilised on selected samples. Protein-A purified antibodies were buffer-exchanged into PBS.

### Preparation of Fcγ Rand FcRn Receptors

### Protocol 1

FcγRIIaH, IIaR, IIb, IIIaF and IIIaV were produced in HEK293-6E cells while FcγRIa was produced in CHO-3E7 cells as described previously (Dorian-Thibaudeau, et al., 2014, J. Immunol. Methods, 408:24-34). The human FcRn was also expressed in HEK293-6E cells by the co-transfection of the alpha subunit (p51) extracellular domain containing a TEV-cleavable C-terminal His-tag with β2-microglobulin in a 1:1 ratio. Following purification as described in Dorion-Thibaudeau et al. (ibid.) the C-terminal His-tag was removed by TEV cleavage.

### Protocol 2

Soluble FcγRI extracellular domain with a C-terminal 6xHis tag was purchased from R&D Systems (Catalogue number 1257-Fc). Soluble FcγRIIaH, IIaR, IIb, IIIaF and IIIaV extracellular domains were produced in HEK293-6E cells with C-terminal 10xHis tags. Cells were transfected in exponential growth phase (1.5 to 2.0 million cells/mL) with 1 μg DNA/mL cells using DNA pre-complexed with a Gemini cationic lipid (Camilleri et al., 2000, Chem. Commun., 1253-1254). Cells were cultured for up to 7 days in a humidified shaking incubator at 37° C. and 5% carbon dioxide in an appropriately sized Erlenmeyer flask. The time of harvest was determined by when the cell viability dropped below 50%. Culture medium was then collected after centrifugation at 2750×g and clarified using a 0.22 μm filter.

The clarified culture medium was buffer-exchanged by dialysis or tangential flow filtration into pH7.7 load buffer containing 25 mM imidazole and applied to a Ni Sepharose 6 column (GE Healthcare, Little Chalfont, U.K.), then eluted by increasing the buffer imidazole concentration to 300 mM. Eluted protein was concentrated and buffer-exchanged into PBS by dia-filtration then further purified by size exclusion chromatography (Superdex® 75 column (GE Healthcare, Little Chalfont, U.K.))

Soluble human FcRn extracellular domain was expressed in HEK 293-6E cells by the co-transfection of the alpha subunit containing a C-terminal 6xHis-tag with β2 microglobulin at a 1:1 ratio and expressed as otherwise described for the FcγRs. The pH of the clarified culture medium was adjusted to pH 5.3 with citrate then loaded onto an IgG Sepharose column (GE Healthcare, Little Chalfont, U.K.). Bound protein was eluted with pH 7.7 HEPES buffer. Eluted protein was concentrated and buffer-exchanged into PBS by dia-filtration then further purified by size exclusion chromatography (Superdex® 75 column (GE Healthcare, Little Chalfont, U.K.))

### Fcγ Receptor Binding (Surface Plasmon Resonance (SPR))

### Protocol 1

Affinity of FcγRs to antibody Fc was measured by SPR using a ProteOn™ XPR36 at 25° C. with PBS containing 150 mM NaCl, 3.4 mM EDTA, and 0.05% Tween 20 at pH 7.4 as the running buffer. For trastuzumab variants, recombinant HER2 was immobilized on a GLM sensorchip using standard amine coupling with a BioRad amine coupling kit. Briefly, the GLM sensorchip was activated with NHS/EDC followed by injecting HER2 at 4.0 μg/mL in 10 mM NaOAc (pH 4.5) until approximately 3000 resonance units (RUs) were immobilized. This was followed by quenching the remaining active groups with ethanolamine. Wild-type trastuzumab variants were indirectly captured onto their SPR surface by injecting a 40 μg/mL solution purified antibody in the ligand direction at 25 μL/min for 240 s resulting in approx. 500 RUs on the surface. Following buffer injections to establish a stable baseline in the analyte direction, analyte was injected at 50 μL/min for 120 s with a 180 s dissociation phase to obtain a set of binding sensorgrams. Five concentrations of a 3-fold dilution series of the FcγRs with 10 μM top nominal concentrations for all receptors were used except 30 nM for FcγR1a, and buffer was included for double referencing. Resultant Kd (affinity) values were determined from the aligned and referenced sensorgrams using the Equilibrium Fit model in ProteOn™ Manager v3.1.0 with reported values as the mean of two or three independent runs.

### Protocol 2

Affinity of FcγRs to antibody Fc was measured by SPR using a Biacore™ 4000 (GE Healthcare, Little Chalfont, U.K.) at 25° C. with PBSTE (PBS with 0.05% Tween-20 and 3.4 mM EDTA) as the running buffer. For anti-HER2 antibodies, a CM5 chip (GE Healthcare, Little Chalfont, U.K.) was immobilized with recombinant HER2 extracellular domain (Merck, Darmstadt, Germany or ThermoFisher Scientific, Loughborough, U.K.) utilizing amine coupling (EDC/NHS chemistry). Briefly, the CM5 sensorchip was activated with NHS/EDC followed by injection of HER2 at 10.0 μg/mL in 10 mM NaOAc (pH 4.5). Immobilization levels ranged between 1000-4000 RU. Any remaining active groups were then quenched with ethanolamine. Antibodies were first captured on the immobilized surface of the chip by injecting at approximately 15 μg/ml across the spots and flow cells for 35 s at a flow-rate of 10 μl/min, leaving spot 3 blank for reference subtraction. Receptors were diluted in PBSTE buffer to a defined concentration range that was dependent on their expected affinity. Six concentrations were used per analyte including zero. Analyte contact time was optimized dependent on the receptor used and its expected kinetics. For example, for FcγRIIB and FcγRIIaR contact time was 18 s at 30 μl/min. The chip surface was regenerated after each analyte concentration injection with 87 mM phosphoric acid. Prior to testing, the chip was prepared with 3×18 s injections of 87 mM phosphoric acid. Double reference subtraction was performed (reference spot 3 and 0 receptor concentration) and binding responses were normalised by the antibody capture level. Samples were analysed using either kinetics and/or steady state (equilibrium) fit models.

### FcRn Binding (Surface Plasmon Resonance (SPR))

### Protocol 1

Affinity of FcRn for antibody variant Fc was measured by SPR using a ProteOn™ XPR36 at 25° C. with HBS-EP+ (10 mM HEPES, 150 mM NaCl, 0.003% M EDTA and 0.05% v/v Surfactant P20 (Teknova, Hollister, U.S.A.)) at pH 7.4 or pH 6.0 as the running buffer. Protein L (ThermoScientific, Loughborough, U.K.) was immobilised on a GLM sensorchip using standard amine coupling with a GE Healthcare coupling kit. Briefly, the GLM sensorchip was activated with NHS/EDC followed by injecting protein L at 50 μg/mL in 10 mM NaOAc (pH 4.5) until approximately 3000 resonance units (RUs) were immobilized, followed by quenching the remaining active groups with ethanolamine. Antibody variants were indirectly captured onto their SPR surface by injecting a 50 μg/mL solution of purified antibody in the ligand direction at 30 L/min for 120 s. Following buffer injections to establish a stable baseline in the analyte direction, analyte was injected at 40 μL/min for 300 s with a 600 s dissociation phase to obtain a set of binding sensorgrams. Five concentrations of a 4-fold dilution series of FcRn with 2048 nM top nominal concentration were used and buffer was included for double referencing. Resultant Kd (affinity) values were determined from the aligned and referenced sensorgrams using the Equilibrium Fit model in ProteOn™ Manager v3.1.0.

### Protocol 2

Affinity of FcRn for antibody variant Fc was measured by SPR using a Biacore™ T200 (GE Healthcare, Little Chalfont, U.K.) at 25° C. with HBS-EP+pH 7.4 or MES pH 6.0 as the running buffer. Samples were captured on an immobilized protein L CM5 chip (GE Healthcare), but 4G7 anti-CD19 antibodies failed to capture. Antibodies were first captured on the immobilized surface of the chip by injecting at approximately 15 μg/ml across the spots and flow cells for 60 s at a flowrate of 5 μl/min. The receptor was diluted to a defined concentration range in HBS-EP+pH 7.4 or MES pH 6.0 buffer. Three concentrations (4096, 512 and 0 nM) were used per analyte at pH 7.4 and four (512, 64, 8 and 0 nM) were used per analyte at pH 6.0. The chip surface was regenerated after each analyte concentration injection with 10 mM Glycine pH 1.5. Results were analysed using Biacore™ T200 Evaluation V2 software and 1:1 binding kinetics model.

### Protocol 3

Affinity of FcRn was measured by SPR using an IBIS MX96 (IBIS Technologies, Enschede, The Netherlands) at 25° C. with HBS-EP+pH 7.4 or MES pH 6.0 as the running buffer. Sample was diluted in pH 4.5 acetate buffer then captured onto a SensEye® G Easy2Spot® sensor chip (SensEye, Enschede, The Netherlands) using a continuous flow microspotter (Carterra, Salt Lake City, USA). The receptor was diluted to a defined concentration range in HBS-EP+pH 7.4 or MES pH 6.0 buffer. Three concentrations (4096, 512 and 0 nM) were used per analyte at pH 7.4 and four (512, 64, 8 and 0 nM) were used per analyte at pH 6.0. The chip surface was regenerated after each analyte concentration injection with 10 mM Glycine pH 2.0. Results were analysed using Scrubber V2 (BioLogic Software, Campbell, Australia) and a kinetic fit model.

### Protocol 4

Antibodies were screened for FcRn binding using a Biacore™ T200 (GE Healthcare) surface plasmon resonance instrument. Experiments were carried out at 25° C. using running buffer containing PBS with 0.05% Tween®20 and 3.4 mM EDTA at pH6. Biotinylated FcRn (produced by Protocol 1 above) was captured onto a CM-5 sensorchip which previously had neutravidin (Thermo Fisher, Waltham MA) immobilized on the blank and capture surfaces using standard amine coupling. Antibody dilutions were then flowed over the FcRn and control surfaces. Using the immobilization wizard within the Biacore control software, 25 ug/mL neutravidin in 10 mM sodium acetate pH 4.5 buffer was added to each NHS/EDC activated surface until 2000 RUs were reached. To create the FcRn surface, the biotinylated FcRn was diluted to 2 ug/mL in PBS containing 0.05% Tween®20 and 3.4 mM EDTA at pH7.4 and injected over the capture surface at a flow rate of 25 ug/mL for 110 seconds until 32 RUs were captured. This FcRn surface was used against all antibodies. Antibodies were screened in duplicate using single cycle kinetics. Five concentrations were injected between 900 and 11.1 nM using a 3-fold dilution in pH6 running buffer at 25 uL/min with a 90 s association and a 180 s dissociation. The FcRn surface was regenerated with a 30 s injection of pH7.4 buffer at 30 uL/min between the different antibody variants. The sensorgrams were double referenced to the blank control surface and fit using the affinity binding model to generate KD values for each antibody-FcRn interaction.

### Differential Scanning Calorimetry

### Protocol 1

Each antibody construct was diluted to 0.2 mg/mL in PBS, and a total of 400 μL was used for DSC analysis with a VP-Capillary DSC (GE Healthcare). At the start of each DSC run, five buffer blank injections were performed to stabilize the baseline, and a buffer injection was placed before each antibody injection for referencing. Each sample was scanned from 20-100° C. at a 60° C./h rate, with low feedback, 8 s filter, 5 min preTstat, and 70 psi nitrogen pressure. The resulting thermograms were referenced and analyzed using Origin 7 software (OriginLab Corporation, Northampton, MA).

### Protocol 2

Antibody constructs were assessed by the same method as described for Protocol 1 above except that antibody concentrations of 0.1-1.0 mg/ml were used, with concentrations of 0.4 mg/ml or greater preferred.

### Differential Scanning Fluorimetry

### Protocol 1

20 μL of purified sample (between 0.2 and 1.0 mg/mL) was added to 10 μL of SYPRO® Orange (Invitrogen, Paisley, U.K.), diluted from 5000× stock to 20× with reverse osmosis (RO) water and placed in a clear walled 96-well PCR plate. Samples were incubated at 40° C. for 5 min, then the fluorescence emission of the SYPRO® Orange was measured using a BioRad CFX Connect™ RT-PCR machine (BioRad, Watford, U.K.) between 40-95° C. using a 15° C./h rate. Bio-Rad CFX Manager™ version 3.1 was used to analyse the peaks and derive temperatures of protein unfolding events which were then correlated to the unfolding of known domains within the protein.

### Protocol 2

10 μL of purified sample (between 0.2 and 1.0 mg/mL) was loaded into Prometheus NT.Plex nanoDSF standard-grade glass capillaries (PR-AC002, NanoTemper Technologies, London, U.K.) and analysed in a Prometheus NT.Plex nanoDSF (NanoTemper Technologies, London, U.K.) between 20-95° C. using a 60° C./h rate. PR.stability Analysis software version 1.02 was used to analyse the peaks and derive temperatures of protein unfolding events which were then correlated to the unfolding of known domains within the protein.

### Size Exclusion Chromatography

10 μL of purified sample (within a concentration range of between 0.2 and 2 mg/mL) was injected onto a Supelco TSKgel® G3000 SWXL size exclusion column (Tosoh, Reading, U.K.) using an Agilent 1100 HPLC system (Agilent, Stockport, U.K.) flowing 400 mM sodium phosphate, 200 mM NaCl, pH 6.8 mobile phase at a constant 1.0 mL/minute with a run time of 15 minutes per sample. A diode array detector was connected in line of the flow after the column and the UV/vis absorption at 210 and 280 nm recorded. The resultant traces were integrated using Chemstation software (Agilent, Stockport, U.K.) and subsequently analyzed using ChromView™ software. Sample purity was recorded by categorization of % area main peak compared to total % area of peaks with a higher molecular weight than main peak and total % area of peaks with a lower molecular weight than main peak.

### Liquid-Chromatography Mass Spectrometry

Mass spectrometry was used to confirm the identity of samples. To remove N-linked glycosylations, 10 μl of 80 μg/ml PNGase F in 5% glycerol was added to 50 μL of antibody sample (within a concentration range of between 0.2 and 2 mg/mL) and the mixture incubated overnight at 30° C. Immediately prior to MS analysis, 5 μL of 0.5M DTT was added to each sample. Using an Agilent 1200 HPLC system (Agilent Technologies, Stockport, U.K.) 3 to 5 μg of sample was injected onto a reverse-phase guard column which was washed with 0.1% formate, 5% acetonitrile then sample was eluted with 0.1% formate, 90% acetonitrile at a flow rate of 0.5 ml/min. The eluate was directed to a 6224 Accurate-Mass TOF LC/MS mass spectrometer (Agilent Technologies) controlled from a PC running MassHunter software (Agilent Technologies). Sample masses were determined by deconvolution of the charge envelopes using MassHunter.

### C1q Binding

Binding of antibody constructs to human C1q was evaluated by ELISA. Test antibody constructs were coated onto wells of a 96-well flat-bottomed Nunc Maxisorp® plate (Invitrogen, Paisley, U.K.) by addition of 100 μl of 10 g/ml test antibody in PBS per well. Plates were sealed and incubated at 4° C. for 16 h. Plates were washed 3 times with 300 μl of PBS containing 0.05% (v/v) Tween®20. The plate surface was then blocked by addition of 200 μl of 1% (w/v) bovine serum albumin per well. Plates were incubated at ambient temperature for 1 h then washed as before. Recombinant human C1q (C1740, Sigma Aldrich, Gillingham, U.K.) was diluted in 50 mM carbonate/bicarbonate buffer (C3041, Sigma Aldrich) to final assay concentrations and 100 μl added per well. Samples were incubated for 2 h at ambient temperature and plates were washed as before. 100 μl of sheep anti-human C1q-HRP (Ab46191, AbCam, Cambridge, U.K.) diluted with PBS to 2 g/ml was then added per well, samples incubated at ambient temperature for 1 h, then plates washed as before. For detection, 100 μl of Sureblue™ TMB (52-00-01, Seracare, Milford, U.S.A.) was added per well and samples incubated with agitation for 20 min at ambient temperature. Reactions were stopped by addition of 100 μl of 1M HCl to each well. Absorbance of each sample well was then measured at 450 nm using a M5e SpectraMax® plate reader (Molecular Devices, Wokingham, U.K.). For each antibody variant 7 C1q concentrations from 2 g/ml to 6 ng/ml in half-log steps plus a no C1q control were tested in duplicate. Data were analysed using Prism (GraphPad, San Diego, U.S.A.). Binding curves were fitted using a 4-parameter non-linear regression model of the absorbance and log-transformed C1q concentration. Concentration of C1q at which binding exceeded a threshold absorbance was interpolated from the fitted curve.

### Example 1: Stability Mutations Identified by in Silico Prediction Tools

Using a three-dimensional structure of FcγRIIb bound to an IgG1 Fc, single mutations were made in silico at each position within the CH2 domain, and at specific positions in the CH3 domain that are within two shells of the CH2 domain. A first shell residue is a residue that interacts directly with the CH2 domain, and a second shell residue is a residue that interacts with at least one first shell residue. Substitutions with all possible amino acids except proline or cysteine were made at each position. Identified mutations that enhance the thermal stability of the Fc are referred to as stability-enhancing mutations.

The stability-enhancing mutations were initially investigated in the context of Fc variants selective for FcγRIIb. As binding of FcγRIIb to the IgG Fc results in an asymmetric complex, only mutations that improved stability in silico for both chains of the Fc were selected for testing to ensure that the stability mutations are compatible with variants selective for FcγRIIb, as well as with other antibody therapeutics.

All models generated in silico were analyzed using a number of molecular modeling tools including in silico mutagenesis and packing modeling with in-house software tools. All models were scored and ranked based on a number of factors including knowledge-based and physics-based potential for folding and complex formation, as well as combined energy contribution of electrostatics, solvent screening, Lennard-Jones and hydrophobic interactions.

Results were further filtered to select the best stability-enhancing mutations that preferably had the following characteristics:
are not solvent exposed (<30% solvent accessible surface area (SASA) favored, <50% accepted)
are distant from the FcγR interface
are not known to negatively impact FcRn binding
are not known to negatively impact C1q binding
do not impact N-glycosylation of the Fc at position N297.

The above process identified the mutations A287F, T289W, A339W, A339Q, A378W and M428F as potential stability-enhancing mutations. Six variants of trastuzumab (Scaffold 1) were constructed as described in the General Methods, each including one of the identified mutations. Each variant was assessed for expression, aggregation, thermal stability and binding affinity for FcγRIIa, FcγRIIb and FcRn as described in the General Methods. Specifically, aggregation was assessed by analytical SEC; thermal stability was assessed by DSC (Protocol 2) and DSF (Protocol 1); FcγRIIa and FcγRIIb binding were assessed by Biacore binding (Protocol 2), and FcRn binding was assessed by Protocol 1. The results are shown in Tables 1.1 and 1.2.

TABLE 1.1
Characteristics of Mutations Identified by in silico Prediction Tools

| | | Relative | Tₘ CH2 | ΔTₘ CH2 | Tₘ CH2 | aSEC | aSEC | aSEC % |
|---|---|---|---|---|---|---|---|---|
| Variant | | SASA¹ | DSC | DSC | DSF | # | Retention | Main |
| # | Mutation | (%) | (° C.) | (° C.)² | (° C.) | Peaks | Time | Peak |
| 16588 | WT | | 71.6 | | 69.0 | 2 | 7.58 | 98.9 |
| 19303 | A287F | 25 | 78.1 | 6.5 | 74.0 | 1 | 7.75 | 100.0 |
| 19304 | T289W | 52 | 66.0 | −5.6 | 70.0 | 1 | 7.78 | 100.0 |
| 19305 | A339W | 39 | 66.3 | −5.3 | 64.5 | 2 | 15.4³ | 99.3 |
| 19306 | A339Q | 39 | 72.5 | 0.9 | 70.0 | 1 | 7.75 | 100.0 |
| 19307 | A378W | 16 | 71.9 | 0.3 | 70.0 | 1 | 7.75 | 100.0 |
| 19308 | M428F | 6 | 76.0 | 4.4 | 72.5 | 1 | 7.73 | 100.0 |

¹The relative SASA is calculated based on the average solvent accessible volumes and area per residue for both chains. The output is relative to fully exposed residue.
²ΔTₘ indicates the difference between the Tₘ mutated − Tₘ wild-type (v16588, WT trastuzumab).
³The data for variant 19305 was generated at a flow-rate of 0.5 ml/min instead of 1.0 ml/min. The retention time therefore is in close agreement in terms of column retention as compared to that of the other variants.

TABLE 1.2
Characteristics of Mutations Identified by in silico Prediction Tools

| | | | | Relative | Relative | FcRn K_{D} | |
|---|---|---|---|---|---|---|---|
| | | FcγRIIb | FcγRIIa | FcγRIIb | FcγRIIa | (nM) | |
| Variant # | Mutation | K_{D} (M) | K_{D} (M) | Binding¹ | Binding¹ | pH 7.4 | pH 6.0 |
| 16588 | WT | 3.80E−06 | 1.10E−06 | — | — | NB² | 641 |
| 19303 | A287F | 4.40E−06 | 1.07E−06 | 0.86 | 1.03 | NB | 690 |
| 19304 | T289W | 3.46E−06 | 1.10E−06 | 1.10 | 1.00 | ND³ | ND |
| 19305 | A339W | 2.10E−06 | 6.40E−07 | 1.81 | 1.72 | ND | ND |
| 19306 | A339Q | 3.81E−06 | 1.04E−06 | 1.00 | 1.05 | ND | ND |
| 19307 | A378W | 5.46E−06 | 1.45E−06 | 0.70 | 0.76 | ND | ND |
| 19308 | M428F | 3.51E−06 | 1.03E−06 | 1.08 | 1.06 | NB | 464 |

¹Change with respect to the wild type expressed as K_{D} wt/K_{D} mut for FcγRIIb and FcγRIIa.
²NB = no binding.
³ND = not determined.

The mutations A287F and M428F were selected for further assessment based on the following criteria:
Tₘ increase by DSC>2° C. for a single point mutation
retention of wild-type like properties (WT value±300%) in terms of FcγRIIb and FcγRIIa binding
retention of binding to FcRn (<2-fold difference in K_{D} relative to WT)
monomeric content >95% by analytical SEC.

The stabilization by the mutation A287F is energetically favorable and likely arises from the creation of stacked Π-Π interactions with position W277 and burying of a hydrogen bond between W277 and S304. As such, alternate mutations at these positions with amino acids that have similar properties in terms of aromaticity and hydrophobicity are predicted to increase stability and thus be stability-enhancing mutations. These include the mutations A287Y, A287W and A287H, as well as A287M. The latter mutation is predicted to bury the hydrogen bond but would likely provide a lower stabilization due to loss of the Π-Π stacking interaction.

FIG. 1B shows the locations of positions A287 and M428 in the IgG Fc region.

### Example 2: Stability Mutations Identified by Bioinformatic Analysis

Sequences from multiple organisms and subtypes of IgG (59 non-redundant sequences extracted from Uniprot) were aligned with ClustalX (Larkin, et al., 2007, Bioinformatics, 23:2947-2948) to differentiate residues that are conserved for folding or function from residues that can be substituted. Non-surface exposed positions with some sequence identity across all IgGs were identified along with alternate common residue(s) found in other species or subtypes. Additional filtering based on relative SASA at the selected position(s) was included to reduce the risk of immunogenicity.

Results were filtered to select the best stability-enhancing mutations that preferably had the following characteristics:
are not solvent exposed (<30% solvent accessible surface area (SASA) favored, <50% accepted)
are partially conserved (sequence identity across all CH₂ IgG<85% favored, <95% accepted)
are distant from the FcγR interface
are not known to negatively impact FcRn binding
are not known to negatively impact C1q binding
do not impact N-glycosylation of the Fc at position N297.

Amino acid substitutions found in mouse IgG2a were favored over other species as the CH2 domain Tₘ is higher in murine IgG2a (Tₘ=˜80° C.) than in human IgG1 (Tₘ=72° C.). The stability of the CH2 domain for other species was not taken into consideration.

The above approach identified the mutations L242I, T250V, F275I, V279I, V308I, Y319F, P247I, M252S, L309Q, L314M and K334R as potential stability-enhancing mutations. Eleven variants of trastuzumab (Scaffold 1) were constructed as described in the General Methods, each including one of the identified mutations. Each variant was assessed for expression in mammalian cells, aggregation post-purification, thermal stability and binding affinity for FcγRIIb, FcγRIIa and FcRn as described in Example 1. The results are shown in Tables 2.1 and 2.2.

TABLE 2.1
Characteristics of Mutations Identified by Bioinformatic Analysis

| | | Rela- | Tₘ | ΔTₘ | Tₘ | | aSEC | aSEC |
|---|---|---|---|---|---|---|---|---|
| | | tive | CH2 | CH2 | CH2 | aSEC | Re- | % |
| Variant | Mu- | SASA¹ | DSC | DSC | DSF | # | tention | Main |
| # | tation | (%) | (° C.) | (° C.)² | (° C.) | Peaks | Time | Peak |
| 16588 | WT | — | 71.6 | — | 69.0 | 2 | 7.58 | 98.9 |
| 19309 | L242I | 12 | 71.7 | 0.1 | 70.5 | 1 | 7.73 | 100 |
| 19310 | T250V | 5 | 81.2 | 9.6 | 75.0 | 1 | 7.74 | 100 |
| 19311 | F275I | 17 | 72.6 | 1.0 | 70.0 | 1 | 7.76 | 100 |
| 19312 | V279I | 14 | 70.8 | −0.8 | 69.5 | 1 | 7.75 | 100 |
| 19313 | V308I | 7 | 73.5 | 1.9 | 70.5 | 1 | 7.75 | 100 |
| 19314 | Y319F | 2 | 69.7 | −1.9 | 69.0 | 1 | 7.75 | 100 |
| 19315 | P247I | 28 | ND³ | ND | ND | 6 | 7.63 | 27.4 |
| 19316 | M252S | 22 | 71.3 | −0.3 | 69.5 | 1 | 7.73 | 100 |
| 19317 | L309Q | 48 | 74.6 | 3.0 | 73.0 | 1 | 7.73 | 100 |
| 19318 | L314M | 24 | 70.4 | −1.2 | 69.0 | 1 | 7.73 | 100 |
| 19319 | K334R | 29 | 72.5 | 0.9 | 70.0 | 1 | 7.76 | 100 |

¹The relative SASA is calculated based on the average solvent accessible volumes and area per residue for both chains. The output is relative to fully exposed residue.
²ΔTₘ indicates the difference between the Tₘ mutated − Tₘ wild-type (v16588, WT trastuzumab).
³ND = not determined.

TABLE 2.2
Characteristics of Mutations Identified by Bioinformatic Analysis

| | | | | | | FcRn K_{D} | |
|---|---|---|---|---|---|---|---|
| | | | | Relative | Relative | (nM) | |
| Variant | Mu- | FcγRIIb | FcγRIIa | FcγRIIb | FcγRIIa | pH | pH |
| # | tation | K_{D} (M) | K_{D} (M) | Binding¹ | Binding¹ | 7.4 | 6.0 |
| 16588 | WT | 3.80E−06 | 1.10E−06 | — | — | NB² | 641 |
| 19309 | L242I | 5.07E−06 | 1.49E−06 | 0.75 | 0.74 | ND³ | ND |
| 19310 | T250V | 3.05E−06 | 8.99E−07 | 1.24 | 1.22 | NB | 821 |
| 19311 | F275I | 2.22E−06 | 7.45E−07 | 1.71 | 1.48 | ND | ND |
| 19312 | V279I | 3.72E−06 | 1.17E−06 | 1.02 | 0.94 | ND | ND |
| 19313 | V308I | 4.03E−06 | 1.24E−06 | 0.94 | 0.89 | ND | ND |
| 19314 | Y319F | 3.49E−06 | 1.10E−06 | 1.09 | 1.00 | ND | ND |
| 19315 | P247I | —⁴ | —⁴ | —⁴ | —⁴ | ND | ND |
| 19316 | M252S | 3.75E−06 | 1.16E−06 | 1.01 | 0.94 | ND | ND |
| 19317 | L309Q | 3.68E−06 | 1.15E−06 | 1.03 | 0.96 | NB | 1000 |
| 19318 | L314M | 5.76E−06 | 2.02E−06 | 0.66 | 0.55 | ND | |
| 19319 | K334R | 2.95E−06 | 9.41E−07 | 1.29 | 1.17 | ND | ND |

¹Change with respect to the wild type expressed as K_{D} wt/K_{D} mut for FcγRIIb and FcγRIIa.
²NB = no binding.
³ND = not determined.
⁴Reliable data for v19315 could not be obtained due to poor capture of the antibody.

The mutations T250V, L309Q and V308I were selected for further assessment based on the following criteria:
Tₘ increase by DSC>2° C. for a single point mutation
retention of wild-type like properties (WT value±300%) in terms of FcγRIIb and FcγRIIa binding
retention of binding to FcRn (<2-fold difference in K_{D} relative to WT)
monomeric content >9500 by analytical SEC.

Alternate mutations at these positions such as T250I, T250A or L309T are predicted to increase stability also due to similar amino acid properties in terms of size and hydrophobicity. Small differences in amino acid size (V vs I or A) and side chain branching (Cβ branched vs non-branched residues) may lead to small variations in the stabilization effect.

FIG. 1B shows the location of position T250 in the IgG Fc region.

### Example 3: Stability Mutations Comprising Non-Natural Disulphide Bonds

Using a three-dimensional structure of FcγRIIb bound to an IgG1 Fe, the distances between all Cα and Cβ atom pairs of the CH2 domain were calculated and averaged for both chains of the Fc in order to determine where a disulphide bond could be introduced. Filtering was based on maximal distances of 7.5 Å for Cα-Cα and 5.0 Å for Cβ-Cβ pairs.

As binding of FcγRIIb to the IgG Fc results in an asymmetric complex, only mutations that improved stability in silico for both chains of the Fc were selected for testing. This selection biases the stability mutations towards mutations that are compatible with variants selective for FcγRIIb and the unbound Fc.

In silico models were created for all possible artificial disulphide bonds and energy-minimized to determine those with the lowest energy. Models were visually inspected and scored based on relative solvent accessible surface area (SASA) of cysteine residues in both chains, backbone and sidechain root-mean-square deviation (RMSD) with respect to the wild-type structure, improvements in knowledge- and physics-based potential for affinity and stability, steric clashes and disulphide strain energy (DSE) score (Katz & Kossiakoff, 1986, J Biol Chem, 261(33):15480-15485).

Results were filtered to select the best stability-enhancing mutations that preferably had the following characteristics:
are not solvent exposed (<30% relative SASA favored, <50% accepted)
are distant from the FcγR interface
are not known to negatively impact FcRn binding
are not known to negatively impact C1q binding
do not impact N-glycosylation of the Fc at position N297
low backbone and sidechain RMSD (RMSD<0.5 Å for backbone and <1.0 Å for sidechains)
equivalent or improved knowledge- and physics-based potential for stability
low DSE scores (<30 (DSE) favored, <50 accepted).

The above process identified the mutation pairs D249C-P257C, F275C-S304C, V263C-V302C, L242C-1336C, T289C-S304C, F243C-T260C, V266C-Y300, V240C-1332C and W277C-V284C as potential stability-enhancing mutations. Thirteen variants of trastuzumab (Scaffold 1) were constructed as described in the General Methods, each including one of the identified pairs of mutations or a mutation pair previously reported in the literature to improve stability through the introduction of a disulphide bond (Jacobsen, et al, 2017, J Biol Chem, 292(5):1865-1875; Gong, et al, 2009, J Biol Chem, 284(21):14203-14210; Gong, et al, 2011, J Biol Chem, 286(31):27288-27293). Each variant was assessed for expression in mammalian cells, aggregation, thermal stability and binding affinity for FcγRIIb, FcγRIIa and FcRn as described in Example 1. The affinity for FcRn receptor was also evaluated in a subset of successful variants. The results are shown in Tables 3.1 and 3.2.

TABLE 3.1
Characteristics of Mutations Introducing Non-Natural Disulfide Bonds

| | | Rel- | Tₘ | ΔTₘ | Tₘ | | aSEC | aSEC |
|---|---|---|---|---|---|---|---|---|
| | | ative | CH2 | CH2 | CH2 | aSEC | Re- | % |
| Variant | | SASA¹ | DSC | DSC | DSF | # | tention | Main |
| # | Mutation | (%) | (° C.) | (° C.)2 | (° C.) | Peaks | Time | Peak |
| 16588 | — | — | 71.6 | 0.0 | 69.0 | 2 | 7.58 | 98.92 |
| 19320 | L242C_K334C | 19.3 | 81.3 | 9.7 | 69.0 | 2 | 7.74 | 97.2 |
| 19321 | A287C_L306C | 21.5 | 76.2 | 4.6 | 72.0 | 1 | 7.75 | 100.0 |
| 19322 | V259C_L306C | 0 | 81.5 | 9.9 | 72.0 | 3 | 7.51 | 67.1 |
| 19323 | R292C_V302C | 16.2 | 67.9 | −3.7 | 72.0 | 1 | 7.71 | 100.0 |
| 19324 | D249C_P257C | 10.7 | 71.2 | −0.4 | 72.0 | 2 | 7.75 | 98.7 |
| 19325 | F275C_S304C | 20.4 | 77.3 | 5.7 | 70.0 | 3 | 7.73 | 82.7 |
| 19326³ | V263C_V302C | 0.9 | 80.0 | 8.4 | 75.0 | 2 | 7.72 | 95.9 |
| 19327 | L242C_I336C | 20.9 | 74.3 | 2.7 | 71.0 | 2 | 7.76 | 98.4 |
| 19328 | T289C_S304C | 39.9 | 68.0 | −3.6 | 69.0 | 2 | 7.75 | 99.0 |
| 19329 | F243C_T260C | 11.1 | 61.1 | −10.5 | 72.0 | 2 | 7.72 | 98.1 |
| 19330 | V266C_Y300C | 2.3 | 66.7 | −4.9 | 72.0 | 1 | 7.64 | 100.0 |
| 19331 | V240C_I332C | 7.7 | 77.6 | 6.0 | 73.0 | 2 | 7.72 | 99.2 |
| 19332 | W277C_V284C | 5.3 | 63.8 | −7.8 | 72.0 | 4 | 7.44 | 34.8 |

¹The relative SASA is calculated based on the average solvent accessible volumes and area per residue for bothchains. The output is relative to fully exposed residue.
²ΔTₘ indicates the difference between the Tₘ mutated − Tₘ wild-type (v16588, WT trastuzumab).
³DSC profile for v19326 was atypical and resulted in ambiguous Tₘ determination for the CH2 domain.

TABLE 3.2
Characteristics of Mutations Introducing Non-Natural Disulfide Bonds

| | | | | | | FcRn K_{D} | |
|---|---|---|---|---|---|---|---|
| | | | Relative | | Relative | (nM) | |
| Variant | | FcγRIIb | FcγRIIb | FcγRIIa | FcγRIIa | pH | pH |
| # | Mutation | K_{D} (M) | Binding¹ | K_{D} (M) | Binding¹ | 7.4 | 6.0 |
| 16588 | — | 3.80E−06 | 1.00 | 1.10E−06 | 1.00 | NB² | 641 |
| 19320 | L242C_K334C | 3.82E−06 | 0.99 | 1.54E−06 | 0.71 | NB | 645 |
| 19321 | A287C_L306C | 3.67E−06 | 1.03 | 1.13E−06 | 0.98 | NB | 693 |
| 19322 | V259C_L306C | 3.72E−06 | 1.02 | 7.58E−07 | 1.45 | ND³ | ND |
| 19323 | R292C_V302C | 1.76E−05 | 0.22 | 3.66E−06 | 0.30 | ND | ND |
| 19324 | D249C_P257C | 1.40E−05 | 0.27 | 3.24E−06 | 0.34 | ND | ND |
| 19325 | F275C_S304C | 4.91E−06 | 0.77 | 1.28E−06 | 0.86 | ND | ND |
| 19326⁴ | V263C_V302C | 2.82E−06 | 1.35 | 9.63E−07 | 1.14 | NB | 604 |
| 19327 | L242C_I336C | 3.22E−06 | 1.18 | 1.14E−06 | 0.97 | ND | ND |
| 19328 | T289C_S304C | 2.05E−06 | 1.85 | 6.89E−07 | 1.60 | ND | ND |
| 19329 | F243C_T260C | 9.83E−06 | 0.39 | 2.93E−06 | 0.38 | ND | ND |
| 19330 | V266C_Y300C | LB⁵ | LB | LB | LB | ND | ND |
| 19331 | V240C_I332C | 1.69E−05 | 0.23 | 6.37E−05 | 0.02 | ND | ND |
| 19332 | W277C_V284C | LB | LB | LB | LB | ND | ND |

¹Change with respect to the wild type expressed as K_{D} wt/K_{D} mut for FcγRIIb and FcγRIIa.
²NB = no binding.
³ND indicates that data was not determined.
⁴Purity profile for v19326 is atypical and results in ambiguous K_{D} determination.
⁵LB = low binding.

Of the identified disulphide bonds, only L242C-1336C was selected for further assessment based on the following criteria:
Tₘ increase by DSC>2° C. for a single pair of mutations
retention of wild-type like properties (WT value±300%) in terms of FcγRIIb and FcγRIIa binding
monomeric content >95% by analytical SEC.

The art-known mutation pairs L242C-K334C, A287C-L306C and V259C-L306C met the following criteria:
Tₘ increase by DSC>2° C. for a single pair of mutations
retention of wild-type like properties (WT value±30%) in terms of FcγRIIb and FcγRIIa binding

The disulphide bond V240C-1332C improved the Tm, but partially abrogated FcγR binding. It is contemplated that this disulphide bond could still be useful in certain contexts where either abrogation of binding is desired or can be mitigated by inclusion of other mutations that promote binding to one or more FcγRs.

### Example 4: Stabilization of FcγRiib Selective Fc Variants

The six best individual mutations (A287F, M428F, T250V, L309Q, L242C_I336C and V308I) identified in the trastuzumab homodimer as described in Examples 1-3 were ported into two different heterodimeric trastuzumab FcγRIIb selective variants (Scaffold 8 and Scaffold 9) to assess their compatibility with other CH2 domain mutations. Additionally, six combinations of two or three stability-enhancing mutations (A287F/M428F, A287F/T250V, M428F/T250V, A287F/M428F/T250V, T250V/L309Q and L242C_I336C/V308I) were tested to assess if an increased stabilization could be obtained by additive or synergistic effects.

Twenty-four variants of trastuzumab in a one-armed antibody format were constructed as described in the General Methods. Each variant included one of two sets of FcγRIIb selectivity-enhancing mutations (Scaffold 8 or Scaffold 9; see Table 4.1) together with the stability-enhancing mutations shown in Tables 4.2 to 4.4. Each variant was assessed for expression, aggregation, thermal stability and binding affinity for FcγRIIb, FcγRIIa and FcγRI as described in Example 1. The results are shown in Tables 4.2 to 4.4.

TABLE 4.1
Parental Variants Used to Assess Compatibility of
Stability and Selectivity-Enhancing Mutations

| | Parental | |
|---|---|---|
| | Variant | |
| Scaffold | # | CH2 Mutations Inducing FcγRIIb Selectivity |
| Scaffold 8 | v27293 | Chain A: G236N_G237A |
| | | Chain B: G236D_G237F_S239D_S267V_H268D_Template 1* |
| Scaffold 9 | v27294 | Chain A: L234F_G236N_H268Q_A327G_A330K_P331S |
| | | Chain B: G236D_S239D_V266L_S267A_H268D |

*Template 1 indicates a replacement of the sequence between positions 325-331 with the sequence: STWFDGGYAT [SEQ ID NO: 1]

A first layer of filtering was applied after purification based on analytical SEC profiles. The area under the curve of the chromatogram was integrated for all signal present and converted to a percentage of each species present in the variant sample. The percentage of high molecular weight (HMW) species observed in the analytical SEC profiles indicates the abundance of full-size antibody formed for each variant using a single DNA ratio for expression. Variants with less than 20% HMW species upon expression at a single DNA ratio were considered successful. Only 3 variants had more than 20% HMW species (see Table 4.2) and were not included in further characterization. Low molecular weight (LMW) species indicates the presence of mis-paired Fc homodimer, which doesn't interfere with determination of the Tm, or with the binding affinity for any of the FcγRs.

TABLE 4.2
Compatibility of Stability and Selectivity-Enhancing Mutations:
Expression & Aggregation

| | | | | | | Re- |
|---|---|---|---|---|---|---|
| | | Stability- | | % | | tention |
| | Parental | Enhancing | % | Hetero- | % | Time |
| Variant | Variant | Mutations | HMW¹ | dimer¹ | LMW¹ | (min)¹ |
| v27293 | v27293² | — | 3.1 | 88.1 | 8.8 | 7.92 |
| v27296 | v27293² | A287F | 2.6 | 91.7 | 5.7 | 7.96 |
| v27298 | v27293² | M428F | 2.8 | 90.1 | 7.1 | 7.95 |
| v27300 | v27293² | T250V | 2.9 | 90.3 | 6.8 | 7.91 |
| v27302 | v27293² | L309Q | 3.5 | 89.7 | 6.8 | 7.96 |
| v27304 | v27293² | L242C_I336C | >20 | — | — | — |
| v27306 | v27293² | V308I | 2.8 | 93.8 | 3.4 | 7.96 |
| v27308 | v27293² | A287F/M428F | 1.7 | 82.0 | 16.3 | 7.96 |
| v27310 | v27293² | A287F/T250V | 1.8 | 86.3 | 11.9 | 7.96 |
| v27312 | v27293² | M428F/T250V | 3.6 | 88.0 | 8.5 | 7.96 |
| v27316 | v27293² | T250V/L309Q | 2.9 | 93.1 | 4.1 | 7.94 |
| v27318 | v27293² | L242C_I336C/ | 19.6 | 75.0 | 5.4 | 7.94 |
| | | V308I | | | | |
| v27294 | v27294³ | — | 3.4 | 94.6 | 1.9 | 7.94 |
| v27297 | v27294³ | A287F | 3.0 | 94.6 | 2.4 | 7.98 |
| v27301 | v27294³ | T250V | 3.0 | 93.2 | 3.8 | 7.93 |
| v27303 | v27294³ | L309Q | 4.8 | 87.2 | 8.0 | 7.95 |
| v27305 | v27294³ | L242C_I336C | 1.6 | 88.6 | 9.9 | 7.98 |
| v27307 | v27294³ | V308I | 4.5 | 89.6 | 5.9 | 7.98 |
| v27309 | v27294³ | A287F/M428F | 2.5 | 88.3 | 9.2 | 7.97 |
| v27311 | v27294³ | A287F/T250V | 2.7 | 89.2 | 8.1 | 7.97 |
| v27313 | v27294³ | M428F/T250V | 9.6 | 75.9 | 14.5 | 7.98 |
| v27317 | v27294³ | T250V/L309Q | 4.6 | 89.5 | 5.9 | 7.96 |
| v27319 | v27294³ | L242C_I336C/ | 2.0 | 86.1 | 11.9 | 8 |
| | | V308I | | | | |

¹% HMW, % heterodimer, % LMW and retention time of monomer all pertain to the profile observed by analytical SEC for each variant and indicates their relative abundance. % HMW corresponds to mis-paired full-size antibody, % heterodimer corresponds to heterodimer one-armed antibody and % LMW corresponds to mis-paired homodimeric Fc or half-antibodies.
²Scaffold 8
³Scaffold 9

TABLE 4.3
Compatibility of Stability and Selectivity-
Enhancing Mutations: Thermal Stability

| | | | | | Theo- |
|---|---|---|---|---|---|
| | Parental | Stability-Enhancing | Tₘ CH₂ | ΔTₘ | retical |
| Variant | Variant | Mutations | (° C.)¹ | (° C.)² | ΔTₘ³ |
| v27293 | v27293⁴ | — | 59.0 | 0.0 | — |
| v27296 | v27293⁴ | A287F | 62.5 | 3.5 | — |
| v27298 | v27293⁴ | M428F | 61.0 | 2.0 | — |
| v27300 | v27293⁴ | T250V | 64.5 | 5.5 | — |
| v27302 | v27293⁴ | L309Q | 61.0 | 2.0 | — |
| v27304 | v27293⁴ | L242C_I336C | N/A⁶ | N/A | N/A |
| v27306 | v27293⁴ | V308I | 59.5 | 0.5 | — |
| v27308 | v27293⁴ | A287F/M428F | 65.5 | 6.5 | 5.5 |
| v27310 | v27293⁴ | A287F/T250V | 68.0 | 9.0 | 9.0 |
| v27312 | v27293⁴ | M428F/T250V | 67.5 | 8.5 | 7.5 |
| v27316 | v27293⁴ | T250V/L309Q | 68.0 | 9.0 | 7.5 |
| v27318 | v27293⁴ | L242C_I336C/V308I | 62.0 | 3.0 | 0.5 |
| v27294 | v27294⁵ | — | 62.0 | 0.0 | — |
| v27297 | v27294⁵ | A287F | 66.0 | 4.0 | — |
| v27301 | v27294⁵ | T250V | 67.5 | 5.5 | — |
| v27303 | v27294⁵ | L309Q | 64.5 | 2.5 | — |
| v27305 | v27294⁵ | L242C_I336C | 62.0 | 0.0 | — |
| v27307 | v27294⁵ | V308I | 63.0 | 1.0 | — |
| v27309 | v27294⁵ | A287F/M428F | 69.0 | 7.0 | 5.0 |
| v27311 | v27294⁵ | A287F/T250V | 71.5 | 9.5 | 9.5 |
| v27313 | v27294⁵ | M428F/T250V | 70.5 | 8.5 | 6.5 |
| v27317 | v27294⁵ | T250V/L309Q | 70.5 | 8.5 | 8.0 |
| v27319 | v27294⁵ | L242C_I336C/V308I | 62.5 | 0.5 | 1.0 |

¹The transition observed for the CH₂ domain by DSF are reported
²ΔTₘ indicates the difference between the Tₘ mutated − Tₘ parental (v27923 or v27924)
³Theoretical ΔTₘ implies an additive stabilization effect based on the point mutation in the respective parental variant
⁴Scaffold 8
⁵Scaffold 9
⁶N/A indicates data was not collected due to low purity of the samples

TABLE 4.4
Compatibility of Stability and Selectivity-Enhancing Mutations:
Binding Affinity for FcγRIIb, FcγRIIa and FcγRI

| | | Stability- | K_{D} | K_{D} | K_{D} | K_{D} |
|---|---|---|---|---|---|---|
| | Parental | Enhancing | FcγRIIb | FcγRIIaR | FcγRIIaH | FcγRI |
| Variant | Variant | Mutations | (M) | (M) | (M) | (M) |
| v27293 | v27293¹ | — | 3.26E−09 | 1.39E−08 | 2.24E−06 | 2.89E−08 |
| v27296 | v27293¹ | A287F | 3.66E−09 | 1.53E−08 | 2.13E−06 | 1.67E−08 |
| v27298 | v27293¹ | M428F | 3.89E−09 | 1.66E−08 | 2.14E−06 | 2.32E−08 |
| v27300 | v27293¹ | T250V | 3.62E−09 | 1.49E−08 | 2.03E−06 | 1.89E−08 |
| v27302 | v27293¹ | L309Q | 2.99E−09 | 1.41E−08 | 2.07E−06 | 1.57E−08 |
| v27304 | v27293¹ | L242C_I336C | N/A³ | N/A | N/A | N/A |
| v27306 | v27293¹ | V308I | 5.19E−09 | 2.02E−08 | 2.38E−06 | 1.30E−08 |
| v27308 | v27293¹ | A287F/M428F | 4.04E−09 | 1.55E−08 | 1.87E−06 | 1.90E−08 |
| v27310 | v27293¹ | A287F/T250V | 3.26E−09 | 1.37E−08 | 1.88E−06 | 1.68E−08 |
| v27312 | v27293¹ | M428F/T250V | 4.50E−09 | 1.77E−08 | 1.96E−06 | 1.97E−08 |
| v27316 | v27293¹ | T250V/L309Q | 4.62E−09 | 1.87E−08 | 2.02E−06 | 2.47E−08 |
| v27318 | v27293¹ | L242C_I336C/ | 5.24E−09 | 1.90E−08 | 2.16E−06 | 5.34E−09 |
| | | V308I | | | | |
| v27294 | v27294² | — | 1.81E−08 | 6.23E−08 | 6.29E−07 | 6.17E−10 |
| v27297 | v27294² | A287F | 2.33E−08 | 7.72E−08 | 6.01E−07 | 6.21E−10 |
| v27301 | v27294² | T250V | 1.93E−08 | 6.85E−08 | 5.50E−07 | 5.69E−10 |
| v27303 | v27294² | L309Q | 2.48E−08 | 7.90E−08 | 5.25E−07 | 7.74E−10 |
| v27305 | v27294² | L242C_I336C | 2.30E−08 | 8.28E−08 | 6.13E−07 | 8.18E−10 |
| v27307 | v27294² | V308I | 2.76E−08 | 8.56E−08 | 6.04E−07 | 8.48E−10 |
| v27309 | v27294² | A287F/M428F | 2.60E−08 | 8.20E−08 | 5.60E−07 | 7.21E−10 |
| v27311 | v27294² | A287F/T250V | 2.41E−08 | 7.94E−08 | 5.67E−07 | 6.79E−10 |
| v27313 | v27294² | M428F/T250V | 1.83E−08 | 6.29E−08 | 5.63E−07 | 7.47E−10 |
| v27317 | v27294² | T250V/L309Q | 2.15E−08 | 7.14E−08 | 5.38E−07 | 7.64E−10 |
| v27319 | v27294² | L242C_I336C/ | 1.55E−08 | 6.14E−08 | 5.75E−07 | 5.32E−10 |
| | | V308I | | | | |

¹Scaffold 8
²Scaffold 9
³N/A indicates data was not collected due to low purity of the samples

Mutations were assessed based on the following criteria:
an increase in Tₘ by DSF>1° C. for a single point mutation and minimally an additive effect when combined
retention of wild-type like properties (<2-fold difference compared to parental variant) in terms of FcγRIIb, FcγRIIa and FcγRI binding
heterodimer content >75% by analytical SEC.

The most effective single mutations based on the above were A287F (+3.5-4° C.), T250V (+5.5° C.), L309Q (+2-2.5° C.) and M428F (+2° C.). V308I as a single mutation also provided a small increase in Tm (+0.5-1.0° C.).

Stability-enhancing designs with either additive or synergistic contributions include A287F/M428F (+6.5-7° C.), A287F/T250V (+9.0-9.5° C.), M428F/T250V (+8.5° C.) and T250V/L309Q (+8.5-9.0° C.). The A287F/M428F, M428/T250V and T250V/L309Q combinations yielded an increase in Tₘ slightly higher than additive effect, while A287F/T250V yielded an additive effect. The combination L242C_I336C/V308I also provided a small increase in Tm over the L242C_I336C mutations alone.

### Example 5: Stabilization of Additional Full-Size Antibody Test Systems

Three of the stability-enhancing designs were each combined with three FcγRIIb selectivity-enhancing designs and transferred into three different full-size antibody systems to assess transferability of the designs across antibodies. The designs were cloned into heterodimeric trastuzumab, anti-CD19 and anti-CD40 antibodies (Scaffolds 3-5) as described in the General Methods. The three FcγRIIb selectivity-enhancing designs are shown in Table 5.1, and the three selected stability-enhancing designs are shown in Tables 5.2-5.5.

TABLE 5.1
Parental Variants Used to Assess Compatibility of
Stability and Selectivity-Enhancing Mutations

| Variant # | CH2 Mutations Inducing FcγRIIb Selectivity |
|---|---|
| v29689 | Chain A: L235F_G236N_G237A |
| | Chain B: G236D_G237F_S239D_S267V_H268D_Template 1.1¹ |
| v29715 | Chain A: L234F_G236N_H268Q_A327G_P329I_A330K_P331S |
| | Chain B: G236D_G237D_S239D_V266L_S267A_H268D |
| v29724 | Chain A: G236N_G237D |
| | Chain B: G236D_G237F_S239D_S267V_H268D_Template 7.1² |

¹“Template 1.1” indicates a replacement of the amino acid residues at positions 325-331 with the following sequence: STWFIGGYAT [SEQ ID NO: 3].
²“Template 7.1” indicates a replacement of the amino acid residues at positions 325-331 with the following sequence: GLDHRGKGYV [SEQ ID NO: 4].

Each variant was assessed for expression in mammalian cells, aggregation post purification and thermal stability as described in Example 1. Binding affinity for FcγRI, FcγRIIb, FcγRIIa and FcγRIIIa for the trastuzumab-based variants was assessed as described in Example 1. C1q binding for the trastuzumab-based variants was assessed as described in the General Methods. Thermal stability was assessed by DSF across multiple antibodies and by DSC for trastuzumab-based variants. The results are shown in Tables 5.2 to 5.6.

Analytical SEC profiles were collected as described in the General Methods with the following modifications for full-size antibody species. The area under the curve of the chromatogram was integrated for all signal present and converted to percentage of each species. The percentage of high molecular weight (HMW) species observed in the aSEC profiles indicates the amount of aggregates formed for each variant upon introduction of the stability-enhancing mutations and was compared to the parental variant across each antibody system. Variants with parental-like properties (+/−5% HMW species) across multiple antibodies were preferred. Lower molecular weight (LMW) species indicate the presence of mis-paired heterodimeric heavy chains, which result in half-antibodies and are due to the use of a non-optimized DNA ratio during expression. The results are shown in Table 5.2.

Purity by LC-MS was evaluated as described in the General Methods to confirm the monomeric content was primarily the desired heterodimer species. The cumulative percentage of half-body A and half-body B indicates the amount of mis-paired homodimers present in each sample for a given DNA ratio used for expression. As the stability-enhancing mutations are symmetric, the CH2 domain Tm, FcRn and C1q binding are not impacted by the presence of mis-paired species. A high content of mis-paired homodimers will however impact the FcγRIIb selectivity observed by SPR. Most samples showed less than 100 mis-paired homodimers. See Table 5.3.

TABLE 5.2
Heterodimer Purity (Analytical SEC)

| | | | | | % | |
|---|---|---|---|---|---|---|
| | Parental | Stability- | | % | Total | % |
| Variant | Selectivity | Enhancing | Anti- | Total | Mono- | Total |
| # | Variant | Mutations | gen | HMW¹ | mer¹ | LMW¹ |
| v21653 | WT | — | HER2 | 0.5 | 97.5 | 2.0 |
| | hetero- | | | | | |
| | dimer² | | | | | |
| v31187 | v29689 | — | HER2 | 0.0 | 100.0 | 0.0 |
| v31274 | v29689 | A287F/T250V | HER2 | 0.5 | 99.5 | 0.0 |
| v31275 | v29689 | M428F/T250V | HER2 | 0.0 | 99.3 | 0.7 |
| v31276 | v29689 | A287F/M428F | HER2 | 0.0 | 100.0 | 0.0 |
| v31256 | v29715 | — | HER2 | 0.7 | 99.3 | 0.0 |
| v31257 | v29715 | A287F/M428F | HER2 | 0.5 | 98.2 | 1.3 |
| v31191 | v29724 | | HER2 | 1.3 | 96.9 | 1.8 |
| v31255 | v29724 | A287F/M428F | HER2 | 0.4 | 98.6 | 1.0 |
| — | v29689 | — | CD19 | 4.0 | 89.5 | 6.6 |
| — | v29689 | A287F/T250V | CD19 | 4.5 | 95.1 | 0.4 |
| — | v29689 | M428F/T250V | CD19 | 4.4 | 90.7 | 4.8 |
| — | v29689 | A287F/M428F | CD19 | 4.7 | 92.3 | 3.1 |
| — | v29715 | — | CD19 | 5.4 | 88.5 | 6.1 |
| — | v29715 | A287F/M428F | CD19 | 4.9 | 82.4 | 12.7 |
| — | v29724 | — | CD19 | 4.4 | 92.9 | 2.6 |
| — | v29724 | A287F/M428F | CD19 | 5.1 | 91.3 | 3.6 |
| — | v29689 | — | CD40 | 4.3 | 93.1 | 2.6 |
| — | v29689 | A287F/T250V | CD40 | 5.9 | 94.2 | 0.0 |
| — | v29689 | M428F/T250V | CD40 | 4.9 | 94.3 | 0.8 |
| — | v29689 | A287F/M428F | CD40 | 4.5 | 95.5 | 0.0 |
| — | v29715 | — | CD40 | 3.5 | 87.1 | 9.4 |
| — | v29715 | A287F/M428F | CD40 | 4.9 | 94.5 | 0.7 |
| — | v29724 | — | CD40 | 10.2 | 70.2 | 19.6 |
| — | v29724 | A287F/M428F | CD40 | 4.0 | 94.6 | 1.4 |

¹% HMW, % monomer, % LMW and retention time of monomer all pertain to the profile observed by analytical SEC for each variant and indicates their relative abundance. % HMW corresponds to aggregates, % monomer corresponds to heterodimer and mispaired homodimer, while % LMW corresponds to half-body.
²Heterodimeric trastuzumab—Scaffold 3.

TABLE 5.3
Heterodimer Purity (LCMS)

| | Parental | | | % | % | % |
|---|---|---|---|---|---|---|
| | Selec- | Stability- | | Total | Half- | Half- |
| Variant | tivity | Enhancing | Anti- | Hetero- | body | body |
| # | Variant | Mutations | gen | dimer | A | B |
| v21653 | WT | — | HER2 | 94.8 | 0.0 | 5.2 |
| | hetero | | | | | |
| | dimer¹ | | | | | |
| v31187 | v29689 | — | HER2 | 99.6 | 0.4 | 0.0 |
| v31274 | v29689 | A287F/T250V | HER2 | 99.3 | 0.7 | 0.0 |
| v31275 | v29689 | M428F/T250V | HER2 | 99.2 | 0.0 | 0.8 |
| v31276 | v29689 | A287F/M428F | HER2 | 99.1 | 0.9 | 0.0 |
| v31256 | v29715 | — | HER2 | 98.0 | 2.0 | 0.0 |
| v31257 | v29715 | A287F/M428F | HER2 | 93.9 | 0.0 | 6.1 |
| v31191 | v29724 | — | HER2 | 92.1 | 0.0 | 7.9 |
| v31255 | v29724 | A287F/M428F | HER2 | 98.6 | 0.0 | 1.4 |
| — | v29689 | — | CD19 | 72.8 | 27.2 | 0.0 |
| — | v29689 | A287F/T250V | CD19 | 100.0 | 0.0 | 0.0 |
| — | v29689 | M428F/T250V | CD19 | 88.9 | 0.0 | 11.1 |
| — | v29689 | A287F/M428F | CD19 | 91.9 | 0.0 | 8.1 |
| — | v29715 | — | CD19 | 85.8 | 0.0 | 14.2 |
| — | v29715 | A287F/M428F | CD19 | 71.0 | 0.0 | 29.0 |
| — | v29724 | — | CD19 | 60.7 | 39.3 | 0.0 |
| — | v29724 | A287F/M428F | CD19 | 92.5 | 0.0 | 7.5 |
| — | v29689 | — | CD40 | 93.9 | 0.0 | 6.1 |
| — | v29689 | A287F/T250V | CD40 | 97.4 | 2.6 | 0.0 |
| — | v29689 | M428F/T250V | CD40 | 98.2 | 0.0 | 1.8 |
| — | v29689 | A287F/M428F | CD40 | 98.3 | 1.7 | 0.0 |
| — | v29715 | — | CD40 | 81.7 | 0.0 | 18.3 |
| — | v29715 | A287F/M428F | CD40 | 91.5 | 8.5 | 0.0 |
| — | v29724 | — | CD40 | 97.6 | 0.0 | 2.4 |
| — | v29724 | A287F/M428F | CD40 | 90.3 | 9.7 | 0.0 |

¹Heterodimeric trastuzumab—Scaffold 3.

TABLE 5.4
Thermal Stability

| | | | | Tm | Δ Tm | Tm | ΔTm |
|---|---|---|---|---|---|---|---|
| | Parental | Stability- | | CH2 | CH2 | CH2 | CH2 |
| Variant | Selectivity | Enhancing | Anti- | DSC | DSC | DSF | DSF |
| # | Variant | Mutations | gen | (° C.) | (° C.)¹ | (° C.) | (° C.)¹ |
| v21653 | WT | — | HER2 | 71.7 | — | 68.5 | — |
| | hetero | | | | | | |
| | dimer² | | | | | | |
| v31187 | v29689 | — | HER2 | 61.8 | 0.0 | 59.0 | 0.0 |
| v31274 | v29689 | A287F/T250V | HER2 | 71.6 | 9.8 | 68.0 | 9.0 |
| v31275 | v29689 | M428F/T250V | HER2 | 70.2 | 8.4 | 67.0 | 8.0 |
| v31276 | v29689 | A287F/M428F | HER2 | 68.0 | 6.1 | 65.0 | 6.0 |
| v31256 | v29715 | — | HER2 | 60.2 | 0.0 | 58.0 | 0.0 |
| v31257 | v29715 | A287F/M428F | HER2 | 67.4 | 7.2 | 64.0 | 6.0 |
| v31191 | v29724 | — | HER2 | 65.0 | 0.0 | 62.5 | 0.0 |
| v31255 | v29724 | A287F/M428F | HER2 | 71.2 | 6.2 | 68.5 | 6.0 |
| — | v29689 | — | CD19 | ND³ | ND | 59.0 | 0.0 |
| — | v29689 | A287F/T250V | CD19 | ND | ND | 67.5 | 8.5 |
| — | v29689 | M428F/T250V | CD19 | ND | ND | 67.0 | 8.0 |
| — | v29689 | A287F/M428F | CD19 | ND | ND | 67.0 | 8.0 |
| — | v29715 | — | CD19 | ND | ND | 57.0 | 0.0 |
| — | v29715 | A287F/M428F | CD19 | ND | ND | 64.0 | 7.0 |
| — | v29724 | — | CD19 | ND | ND | 63.0 | 0.0 |
| — | v29724 | A287F/M428F | CD19 | ND | ND | 68.0 | 5.0 |
| — | v29689 | — | CD40 | ND | ND | 59.0 | 0.0 |
| — | v29689 | A287F/T250V | CD40 | ND | ND | 68.5 | 9.5 |
| — | v29689 | M428F/T250V | CD40 | ND | ND | 68.0 | 9.0 |
| — | v29689 | A287F/M428F | CD40 | ND | ND | 65.0 | 6.0 |
| — | v29715 | — | CD40 | ND | ND | 57.5 | 0.0 |
| — | v29715 | A287F/M428F | CD40 | ND | ND | 64.0 | 6.5 |
| — | v29724 | — | CD40 | ND | ND | 62.5 | 0.0 |
| — | v29724 | A287F/M428F | CD40 | ND | ND | 68.5 | 6.0 |

¹ΔTm indicates the difference between the Tₘ mutated − Tₘ parental (v29689, v29715 or v29724) for each design evaluated by DSC or DSF as indicated.
²Heterodimeric trastuzumab—Scaffold 3.
³ND = not determined

TABLE 5.5
FcγR Binding for Trastuzumab-based Variants¹

| | Parental | | | K_{D} | K_{D} | Relative | | Relative |
|---|---|---|---|---|---|---|---|---|
| | Selec- | Stability- | K_{D} | (M) | (M) | K_{D} | K_{D} | K_{D} |
| Variant | tivity | Enhancing | (M) | FcγRII | FcγRII | (M) | (M) | (M) |
| # | Variant | Mutations | FcγRI | aH | aR | FcγRIIa² | FcγRIIb | FcγRIIb² |
| v21653 | WT | — | 8.35E− | 3.02E− | 4.18E− | 1.00 | 1.59E−06 | 1 |
| | hetero- | | 11 | 07 | 07 | | | |
| | dimer³ | | | | | | | |
| v31187 | v29689 | — | NB⁴ | 9.30E− | 5.54E− | 7.54 | 3.52E−09 | 451 |
| | | | | 06 | 08 | | | |
| v31274 | v29689 | A287F/ | 1.61E− | 7.52E− | 5.11E− | 8.19 | 3.62E−09 | 439 |
| | | T250V | 07 | 06 | 08 | | | |
| v31275 | v29689 | M428F/ | NB | 8.23E− | 4.74E− | 8.82 | 2.95E−09 | 539 |
| | | T250V | | 06 | 08 | | | |
| v31276 | v29689 | M428F/ | NB | 1.04E− | 4.95E− | 8.44 | 3.31E−09 | 479 |
| | | T250V | | 05 | 08 | | | |
| v31256 | v29715 | — | 7.48E− | 1.44E− | 1.04E− | 4.00 | 1.26E−08 | 126 |
| | | | 09 | 05 | 07 | | | |
| v31257 | v29715 | M428F/ | 7.20E− | 1.34E− | 1.01E− | 4.15 | 1.15E−08 | 138 |
| | | T250V | 09 | 05 | 07 | | | |
| v31191 | v29724 | — | NB | NB | 4.68E− | 0.89 | 4.64E−08 | 34 |
| | | | | | 07 | | | |
| v31255 | v29724 | A287F/ | NB | NB | 4.39E− | 0.95 | 3.70E−08 | 43 |
| | | M428F | | | 07 | | | |

¹None of the variants showed any detectable binding to FcγRIIIaV and FcγRIIIaF by SPR.
²Change with respect to the wild type expressed as K_{D} wt/K_{D} mut for FcγRIIb and FcγRIIa.
³Heterodimeric trastuzumab—Scaffold 3.
⁴NB = No binding observed.

TABLE 5.6
C1q binding for Trastuzumab-based Variants

| | Parental | Stability- | C1q | Relative C1q | Relative C1q |
|---|---|---|---|---|---|
| | Selectivity | Enhancing | Binding | binding | binding |
| Variant # | Variant | Mutations | (μg/ml)² | (% WT)² | (% WT)³ |
| v21653 | WT | — | 0.138 | 100 | 100 |
| | heterodimer¹ | | | | |
| v31187 | v29689 | — | 0.017 | 896 | 160 |
| v31274 | v29689 | A287F/T250V | 0.016 | 857 | 162 |
| v31275 | v29689 | M428F/T250V | 0.019 | 739 | 161 |
| v31276 | v29689 | M428F/A287F | 0.016 | 888 | 160 |
| v31256 | v29715 | — | NB⁴ | 0 | 8 |
| v31257 | v29715 | M428F/T250V | NB | 0 | 8 |
| v31191 | v29724 | — | NB | 0 | 24 |
| v31255 | v29724 | A287F/M428F | NB | 0 | 18 |

¹Heterodimeric trastuzumab - Scaffold 3.
²C1q binding affinity calculated from the concentration of C1q at which binding signal exceeded 0.5 absorption, as interpolated from the curve fits.
³C1q binding affinity as calculated from the assay absorbance at 2 μg/ml.
²NB = no binding detected.

All three stability enhancing designs tested (A287F/T250V, M428F/T250V and A287F/M428F) successfully increased the thermal stability by 6-10° C. while maintaining parental-like properties in all other aspects evaluated. Specifically, the designs met the following criteria:
an increase in Tₘ by DSF>5° C. across all 3 antibodies.
retention of wild-type like properties (<2-fold difference with parental variant) in terms of FcγRI, FcγRIIaH, FcγRIIaR, FcγRIIb, FcγRIIIa and C1q biding.
heterodimer content equivalent or better than parental variant by LC-MS.
Monomeric content similar to, or better than, parental variant by aSEC.

### Example 6: FcRn Binding for Full-Size Antibodies Comprising Stability-Enhancing Designs

Three of the stability-enhancing designs were each combined with three FcγRIIb selectivity-enhancing designs and transferred into three different full-size antibody systems (trastuzumab, an anti-CD19 antibody and an anti-CD4 antibody; Scaffolds 3-5) as described in Example 5. The resulting variants were assessed for FcRn binding as described in the General Methods (Protocol 2 for Scaffolds 3/5 and Protocol 3 for Scaffold 3-5). The results are shown in Table 6.1.

TABLE 6.1
FcRn Binding of Antibodies Containing Stability-Enhancing and FcγRIIb
Selectivity-Enhancing Designs

| | Parental | Stability- | | FcRn | FcRn | Relative | Relative |
|---|---|---|---|---|---|---|---|
| Variant | Selectivity | Enhancing | Anti- | K_{D} | K_{D} | K_{D} | K_{D} |
| # | Variant | Mutations | gen | (M)² | (M)³ | (M)² | (M)³ |
| v21653 | WT | — | HER2 | 3.14E−07 | 2.66E−07 | — | — |
| | hetero- | | | | | | |
| | dimer¹ | | | | | | |
| v31187 | v29689 | — | HER2 | 4.24E−07 | 3.31E−07 | 1.00 | 1.00 |
| v31274 | v29689 | A287F/T250V | HER2 | 4.05E−07 | 2.43E−07 | 1.05 | 1.36 |
| v31275 | v29689 | M428F/T250V | HER2 | 4.28E−07 | 1.63E−07 | 0.99 | 2.03 |
| v31276 | v29689 | A287F/M428F | HER2 | 5.03E−07 | 7.30E−07 | 0.84 | 0.45 |
| v31256 | v29715 | — | HER2 | 4.55E−07 | 3.62E−07 | 1.00 | 1.00 |
| v31257 | v29715 | A287F/M428F | HER2 | 2.91E−07 | 3.77E−07 | 1.56 | 0.96 |
| v31191 | v29724 | | HER2 | 2.86E−07 | —⁵ | 1.00 | — |
| v31255 | v29724 | A287F/M428F | HER2 | 2.93E−07 | 3.57E−07 | 0.98 | — |
| — | v29689 | — | CD19 | ND | 7.29E−08 | — | 1.00 |
| — | v29689 | A287F/T250V | CD19 | ND⁴ | 7.84E−08 | — | 0.93 |
| — | v29689 | M428F/T250V | CD19 | ND⁴ | 8.07E−08 | — | 0.90 |
| — | v29689 | A287F/M428F | CD19 | ND⁴ | 7.04E−08 | — | 1.04 |
| — | v29715 | — | CD19 | ND⁴ | 8.13E−08 | — | 1.00 |
| — | v29715 | A287F/M428F | CD19 | ND⁴ | 8.92E−08 | — | 0.91 |
| — | v29724 | — | CD19 | ND⁴ | 1.04E−07⁵ | — | 1.005 |
| — | v29724 | A287F/M428F | CD19 | ND | 8.30E−08 | — | 1.25 |
| — | v29689 | — | CD40 | 5.27E−07 | 1.28E−07 | 1.00 | 1.00 |
| — | v29689 | A287F/T250V | CD40 | 6.17E−07 | 8.49E−08 | 0.85 | 1.51 |
| — | v29689 | M428F/T250V | CD40 | 4.64E−07 | 1.04E−07 | 1.14 | 1.23 |
| — | v29689 | A287F/M428F | CD40 | 3.66E−07 | 7.29E−08 | 1.44 | 1.76 |
| — | v29715 | — | CD40 | 4.43E−07 | 1.26E−07 | 1.00 | 1.00 |
| — | v29715 | A287F/M428F | CD40 | 2.57E−07 | 9.13E−08 | 1.72 | 1.38 |
| — | v29724 | — | CD40 | 4.84E−07 | —⁵ | 1.00 | — |
| — | v29724 | A287F/M428F | CD40 | 3.09E−07 | 8.95E−08 | 1.57 | — |

¹Heterodimeric trastuzumab—Scaffold 3.
²K_{D} determined by Protocol 2 in General Methods. Fold change with respect to the parental variant expressed as K_{D} wt/KD mut
³K_{D} determined by Protocol 3 in General Methods. Fold change with respect to the parental variant expressed as K_{D} wt/KD mut
⁴ND = not determined as the antibody did not bind protein L.
⁵v29724 behaved in a non-standard manner when assessed by Protocol 3 (General Methods). Binding assessed by Protocol 2 (General Methods) was comparable to the other variants.

For all antibodies tested, the stability-enhancing designs did not alter the FcRn binding compared to the respective parental variants (K_{D}<3-fold of parental variants) indicating that the designs are transferable across different antibodies.

### Example 7: Compatibility of Stability-Enhancing Designs with Other Mutations Causing Stability Loss

In order to further assess the transferability and compatibility of three of the best performing stability-enhancing designs (A287F/T250V, M428F/T250V and A287F/M428F), these designs were each combined with the following three sets of CH2 or CH3 mutations (Scaffolds 3, 6 and 7; see General Methods):
Scaffold 3: asymmetric mutations in the CH3 domain to promote heterodimeric Fc formation.
Scaffold 6: N297A mutation which produces aglycosylated antibodies and abrogates the effector function of antibodies. Introduction of the N297A mutation reduces the thermal stability of the variant antibody by 10° C. compared to the wild-type antibody.
Scaffold 7: S2391D/I332E mutations which increase the affinity of the antibody for the FcγRIIIa receptor. Introduction of the S2391D/I332E mutations reduces the thermal stability of the variant antibody by 20° C. compared to the wild-type antibody.

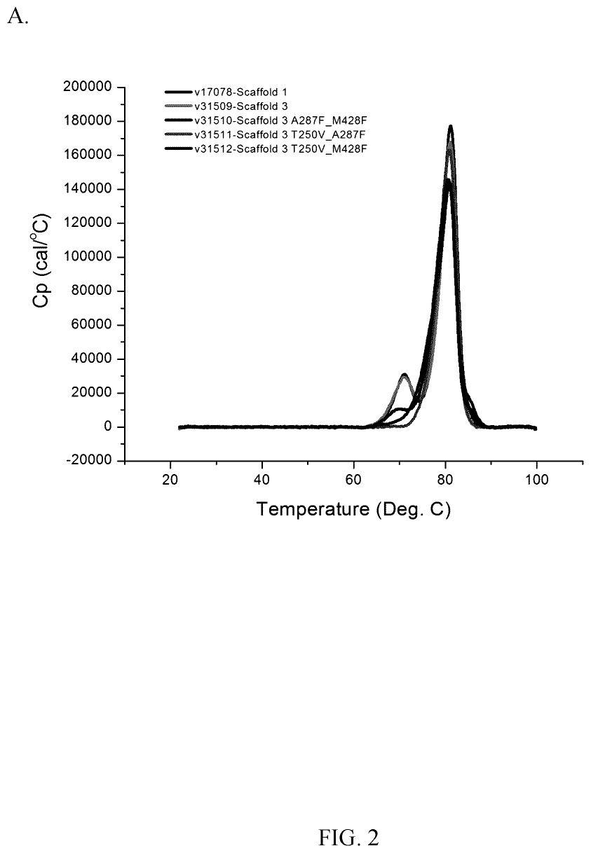

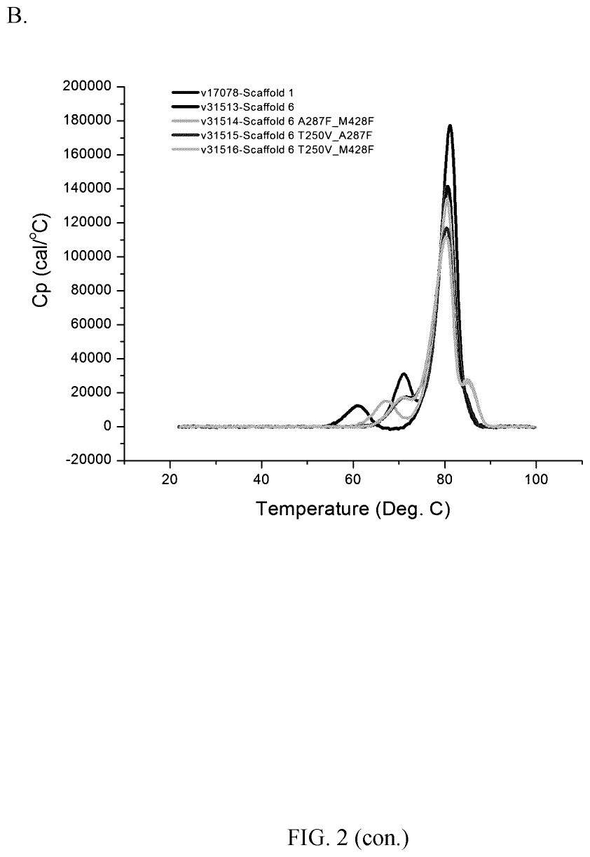

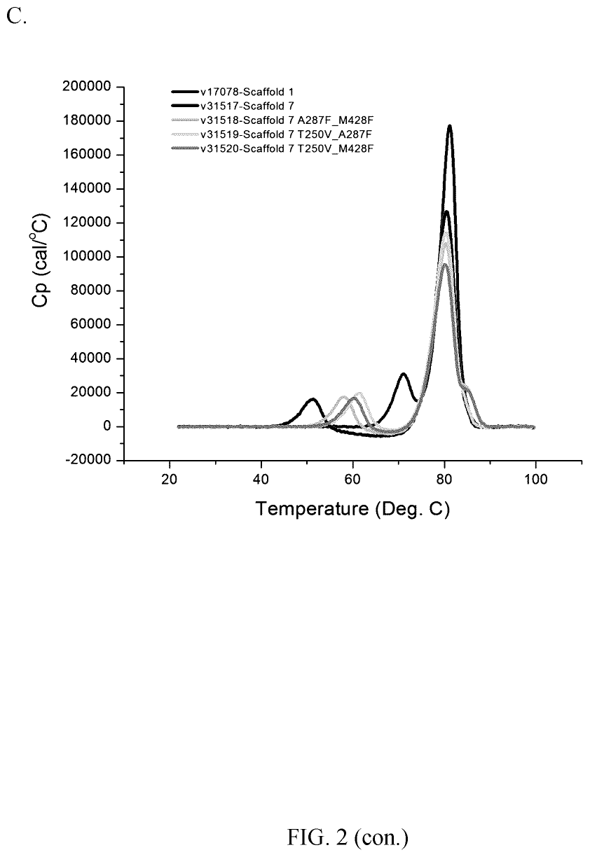

The respective parental variants and mutants were cloned into a trastuzumab scaffold as described in the General Methods. Each variant was assessed for expression in mammalian cells (Protocol 1), thermal stability, binding affinity for FcγRI, FcγRIIb, FcγRIIa and FcγRIIIa (Protocol 1) and FcRn binding (Protocol 4) as described in the General Methods. Thermal stability was assessed by DSC (Protocol 1). The results are shown in Tables 7.1 to 7.4, and in FIG. 2A-C.

TABLE 7.1
Effect of Stability-Enhancing Mutations on FcγRI and
FcγRIII Binding in Different Antibody Scaffolds

| | | Stability- | K_{D} | | K_{D} | |
|---|---|---|---|---|---|---|
| | Descrip- | Enhancing | FcγRI | | FcγRIII | |
| Variant | tion | Mutations | (M) | Fold¹ | (M) | Fold¹ |
| v17078 | WT | — | 8.94E−11 | — | 1.48E−06 | — |
| v31509 | Scaffold 3 | — | 6.66E−11 | 1.0 | 1.19E−06 | 1.0 |
| v31510 | Scaffold 3 | A287F_M428F | 5.39E−11 | 1.2 | 1.07E−06 | 1.1 |
| v31511 | Scaffold 3 | A287F_T250V | 6.77E−11 | 1.0 | 1.07E−06 | 1.1 |
| v31512 | Scaffold 3 | M428F_T250V | 7.95E−11 | 0.8 | 1.23E−06 | 1.0 |
| v31513 | Scaffold 6 | — | 2.57E−08 | 1.0 | NB | — |
| v31514 | Scaffold 6 | A287F_M428F | 1.62E−08 | 1.6 | NB | — |
| v31515 | Scaffold 6 | A287F_T250V | 1.87E−08 | 1.4 | NB | — |
| v31516 | Scaffold 6 | M428F_T250V | 2.00E−08 | 1.3 | NB | — |
| v31517 | Scaffold 7 | — | 1.49E−11 | 1.0 | 3.20E−08 | 1.0 |
| v31518 | Scaffold 7 | A287F_M428F | 1.36E−11 | 1.1 | 3.09E−08 | 1.0 |
| v31519 | Scaffold 7 | A287F_T250V | 1.08E−11 | 1.4 | 2.90E−08 | 1.1 |
| v31520 | Scaffold 7 | M428F_T250V | 1.45E−11 | 1.0 | 3.21E−08 | 1.0 |

¹Fold change with respect to the parental variant expressed as K_{D} wt/K_{D} mut
²NB = no binding detected.

TABLE 7.2
Effect of Stability-Enhancing Mutations on FcγRII Binding in
Different Antibody Scaffolds

| | | Stability- | K_{D} | | K_{D} | | K_{D} | |
|---|---|---|---|---|---|---|---|---|
| | Descrip- | Enhancing | FcγRIIaH | | FcγRIIaR | | FcγRIIb | |
| Variant | tion | Mutations | (M) | Fold¹ | (M) | Fold¹ | (M) | Fold¹ |
| v17078 | WT | — | 1.49E−06 | — | 2.07E−06 | — | 6.85E−06 | — |
| v31509 | Scaffold 3 | — | 1.15E−06 | 1.0 | 1.52E−06 | 1.0 | 4.20E−06 | 1.0 |
| v31510 | Scaffold 3 | A287F_M428F | 1.07E−06 | 1.1 | 1.40E−06 | 1.1 | 3.85E−06 | 1.1 |
| v31511 | Scaffold 3 | A287F_T250V | 1.04E−06 | 1.1 | 1.28E−06 | 1.2 | 3.31E−06 | 1.3 |
| v31512 | Scaffold 3 | M428F_T250V | 9.59E−07 | 1.2 | 1.26E−06 | 1.2 | 3.52E−06 | 1.2 |
| v31513 | Scaffold 6 | — | NB² | — | NB | — | NB | — |
| v31514 | Scaffold 6 | A287F_M428F | NB | — | NB | — | NB | — |
| v31515 | Scaffold 6 | A287F_T250V | NB | — | NB | — | NB | — |
| v31516 | Scaffold 6 | M428F_T250V | NB | — | NB | — | NB | — |
| v31517 | Scaffold 7 | — | 7.75E−07 | 1.0 | 7.75E−07 | 1.0 | 1.25E−06 | 1.0 |
| v31518 | Scaffold 7 | A287F_M428F | 6.71E−07 | 1.2 | 6.91E−07 | 1.1 | 1.15E−06 | 1.1 |
| v31519 | Scaffold 7 | A287F_T250V | 6.55E−07 | 1.2 | 6.85E−07 | 1.1 | 1.14E−06 | 1.1 |
| v31520 | Scaffold 7 | M428F_T250V | 6.44E−07 | 1.2 | 6.67E−07 | 1.2 | 1.13E−06 | 1.1 |

¹Fold change with respect to the parental variant expressed as K_{D} wt/K_{D} mut
²NB = no binding detected.

TABLE 7.3
Effect of Stability-Enhancing Mutations on FcRn
Binding in Different Antibody Scaffolds

| | | Stability- | | |
|---|---|---|---|---|
| | | Enhancing | K_{D} FcRn | |
| Variant | Description | Mutations | (M) | Fold¹ |
| v17078 | WT | — | 2.92E−07 | — |
| v31509 | Scaffold 3 | — | 2.84E−07 | 1.0 |
| v31510 | Scaffold 3 | A287F_M428F | 2.63E−07 | 1.1 |
| v31511 | Scaffold 3 | A287F_T250V | 4.40E−07 | 0.6 |
| v31512 | Scaffold 3 | M428F_T250V | 2.90E−07 | 1.0 |
| v31513 | Scaffold 6 | — | 4.67E−07 | 1.0 |
| v31514 | Scaffold 6 | A287F_M428F | 3.69E−07 | 1.3 |
| v31515 | Scaffold 6 | A287F_T250V | 5.53E−07 | 0.8 |
| v31516 | Scaffold 6 | M428F_T250V | 3.60E−07 | 1.3 |
| v31517 | Scaffold 7 | — | 3.37E−07 | 1.0 |
| v31518 | Scaffold 7 | A287F_M428F | 2.75E−07 | 1.2 |
| v31519 | Scaffold 7 | A287F_T250V | 4.70E−07 | 0.7 |
| v31520 | Scaffold 7 | M428F_T250V | 2.86E−07 | 1.2 |

¹Fold change with respect to the parental variant expressed as K_{D} wt/K_{D} mut

TABLE 7.3
Effect of Stability-Enhancing Mutations on Thermal
Stability (Tm) in Different Antibody Scaffolds

| | | Stability- | | |
|---|---|---|---|---|
| | | Enhancing | Tₘ by DSC | |
| Variant | Description | Mutations | (° C.) | ΔTₘ (° C.) |
| v17078 | WT | — | 71.2 | — |
| v31509 | Scaffold 3 | — | 70.9 | — |
| v31510 | Scaffold 3 | A287F_M428F | 80+ ¹ | >8¹ |
| v31511 | Scaffold 3 | A287F_T250V | 80+ ¹ | >8¹ |
| v31512 | Scaffold 3 | M428F_T250V | 80+ ¹ | >8¹ |
| v31513 | Scaffold 6 | — | 61.2 | — |
| v31514 | Scaffold 6 | A287F_M428F | 66.9 | 5.7 |
| v31515 | Scaffold 6 | A287F_T250V | 71 | 9.9 |
| v31516 | Scaffold 6 | M428F_T250V | 70.7 | 9.5 |
| v31517 | Scaffold 7 | — | 51.3 | — |
| v31518 | Scaffold 7 | A287F_M428F | 58.2 | 6.9 |
| v31519 | Scaffold 7 | A287F_T250V | 61.7 | 10.4 |
| v31520 | Scaffold 7 | M428F_T250V | 60.1 | 8.8 |

¹CH2 transition overlapped with the Fab and CH3 transitions and could not be accurately determined

All three stability-enhancing designs tested (A287F/T250V, M428F/T250V and A287F/M428F) successfully increased the thermal stability of each of the tested scaffolds by 6-10° C. while maintaining parental-like properties in all other aspects evaluated. Specifically, all three designs met the following criteria:
an increase in Tm by DSC>5° C. across all 3 scaffolds
retention of wild-type like properties (<2-fold difference with parental variant) in terms of FcγRI, FcγRIIaH, FcγRIIaR, FcγRIIb, FcγRIIIa and FcRn binding.

Thus, the stability-enhancing designs are compatible with and capable of stabilizing mutations in the CH2 and CH3 domains that alter functional and biological properties of antibodies.

The above sets of CH2 or CH3 mutations (exemplified by Scaffolds 3, 6 and 7) are examples of mutations included in therapeutic molecules currently being evaluated in the clinic (Saxena et al., 2016, Front Immunol, 7:580). Other CH2 and/or CH3 mutations that impact antibody function and stability, such as knobs-into-holes (Ridgeway et al., 1996, Protein Eng., 9:617-621), electrostatic steering (Gunasekaran et al, 2010, JBC, 285, 19637-19646), or others known in the art, are expected to also be compatible with and stabilized by the stability-enhancing mutations.

### Example 8: Compatibility of Stability-Enhancing Designs with Other Immunoglobulin Classes

The sequence and topology of IgG, IgA, IgD, IgE and IgM constant domains were assessed to determine whether the most effective of the stability-enhancing designs identified above could be transferred to other immunoglobulin (Ig) classes and/or subtypes.

The various Ig classes have different functions and biological activities but share a common topology. IgG, IgA, IgD, IgE and IgM are all composed of heavy and light chains. IgG, IgA and IgD constant regions contain CH1, CH2 and CH3 domains that share a common Ig fold suggesting that mutations increasing the stability of IgG could be transferable to the IgA and IgD classes. IgE and IgM differ from the other Ig classes and are composed of CH1, CH2, CH3 and CH4 domains. Based on sequence identity, the CH3 and CH4 domains of IgM and IgE can be considered to be equivalent to the CH2 and CH3 domains of the other Ig classes (see FIGS. 3A and 3B). Review of the structures of the IgG, IgA and IgM Ig domains obtained from the Protein Data Bank (PDB) (PDB ID: 2QEJ, 2WAH and 6KXS, respectively) indicated that, from a structural perspective, these domains have similar folding suggesting that mutations that increase the stability of IgG could be transferable to the IgM class, as well as the IgE class.

Further analysis of the specific interactions of each of the T250V, A287F and M428F mutations within the CH2 and CH3 domains provided further support for the proposition that the stability-enhancing mutations should be transferable to other Ig classes.

Residue T250 is located in a helical region of IgG1 close to the FcRn binding site of the CH2 domain and spatially near to the CH3 domain. A threonine residue is conserved across all IgG subtypes at this position and is substituted by a similar polar residue (serine) in IgM and a charged residue (aspartic acid) in IgA, IgD and IgE (see FIG. 3A). Structural analysis of this region in IgA, IgM and IgG indicated that the helix in IgA and IgM is less buried (PDB ID: 2QEJ and 6KXS) than in IgG1 (PDB ID: 2WAH). As such, mutation of a charged or polar residue at the position equivalent to T250 in other Ig classes to a smaller hydrophobic residue could improve packing of the first helix (246-254) against the second helix (309-316) and the junction of the CH2-CH3 domains, resulting in a more compact structure with an increased buried interface. This in turn could stabilize the CH2 domain against thermal denaturation. Thus, the stability-enhancing mutation T250V is predicted to be effective in increasing the stability of the CH2 domain of IgA, IgD and IgG antibodies and of the CH3 domain of IgE and IgM antibodies.

Residue A287 is located in an exposed β-strand region on the outside of the Ig-fold of the CH2 domain of IgG1. An alanine residue is conserved at position 287 across all IgG subtypes as well as IgA, but substituted by residues such as valine, histidine and threonine in IgD, IgE and IgM. Regardless of the different residues present at this position, however, the local environment and fold is similar across all Ig classes for which structures are available. As described in Example 1, the stabilization by the mutation A287F in IgG1 is energetically favorable and likely arises from the creation of stacked Π-Π interactions with position W277 and burying of a hydrogen bond between positions W277 and S304. Residue W277 is conserved across all Ig classes and residue S304 is conserved across all Ig except IgM (see FIG. 3A and FIG. 3B). As such, the A287F mutation is also predicted to be effective in increasing the stability of the CH2 domain of IgA, IgD and IgG antibodies and of the CH3 domain of IgE and IgM antibodies.

Residue M428 is located in an exposed β-strand region of the CH3 domain of IgG1 and spatially at the interface with the CH2 domain. A methionine at position 428 is conserved across all IgG subtypes, while other Ig classes contain smaller residues at this position such as glycine, valine, alanine or serine (see FIGS. 3A and 3B). Introduction of a larger residue such as phenylalanine at this position likely buries the hydrophobic side chain against the helix from the CH2 domain, with the resulting increase in buried surface at the junction of the CH2-CH3 domains reducing the flexibility and increasing the stability of the CH2 domain. The local structural environment in IgA and IgM is similar to IgG in this region and introduction of a large aromatic residue such as phenylalanine, tyrosine or tryptophan is expected to form additional stacked Π-Π interactions with the surrounding aromatic residues. The mutation M428F is therefore also predicted to increase the stability of the CH2 domain of IgA, IgD and IgG antibodies and of the CH3 domain of IgE and IgM antibodies.

As demonstrated in Examples 4-7, the T250V, A287F and M428F stability-enhancing mutations are compatible as pairs and can be combined to yield an additive stabilization effect. A similar compatibility is expected across other Ig classes for these stability-enhancing designs (A287F/T250V, M428F/T250V and A287F/M428F).

### Example 9: Effect of Stability Mutations on Aggregation Propensity Under Stress Conditions

To assess whether the increased thermal stability achieved by incorporation of the stability-enhancing mutations could reduce the aggregation propensity of antibodies, fifteen antibodies with variant Fc regions having a range of thermal stabilities were prepared with or without the T250V/A287F stability-enhancing design. The resulting antibody variants were then submitted to a stress experiment in which they were evaluated for change in aggregate proportion following a 2-week incubation at 40° C. under either neutral or mildly acidic conditions.

Each antibody variant was based on Scaffold 3 and included various combinations of mutations in the CH2 domain as shown in Table 9.1. The respective parental variants and stability mutants were cloned into Scaffold 3 as described in the General Methods. Each variant was assessed for expression in mammalian cells (Protocol 2), aggregation by size exclusion chromatography and thermal stability by DSF (Protocol 2). The results are shown in Tables 9.1 and 9.2.

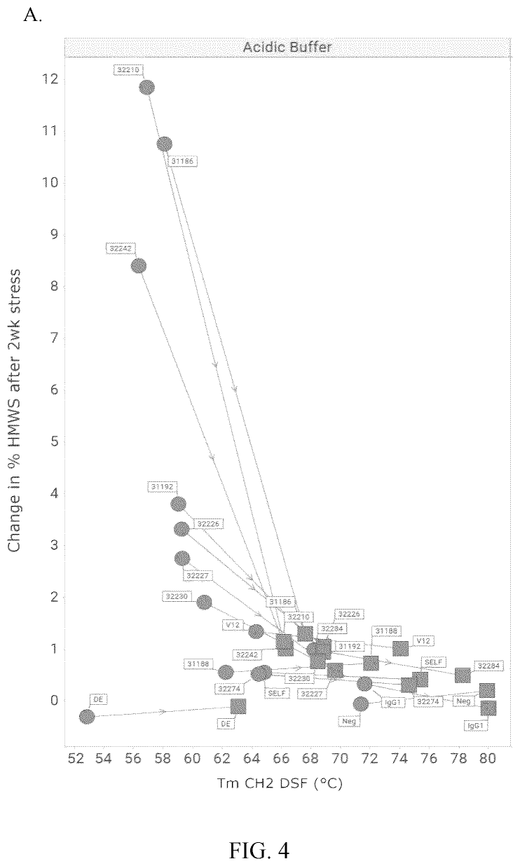

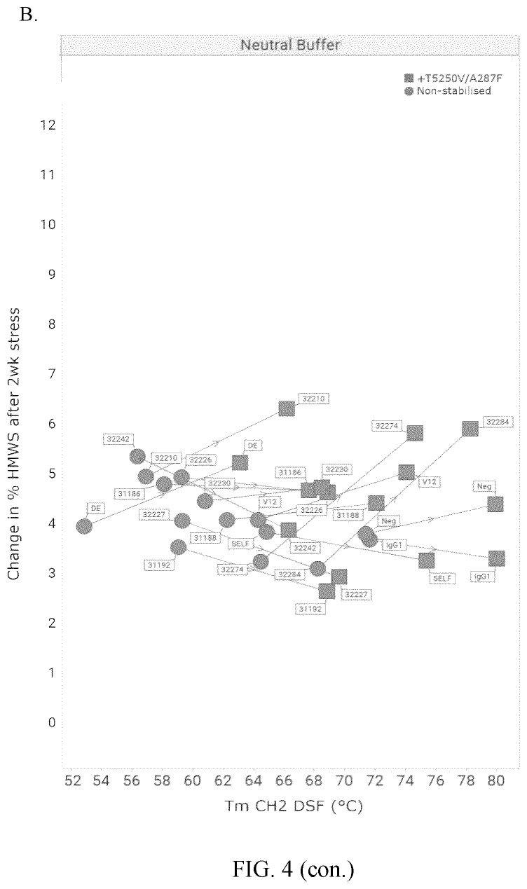

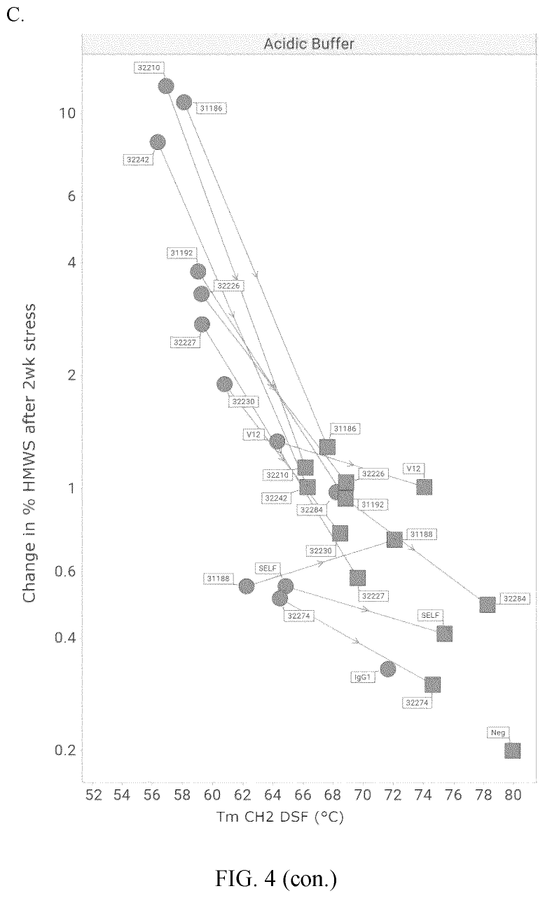

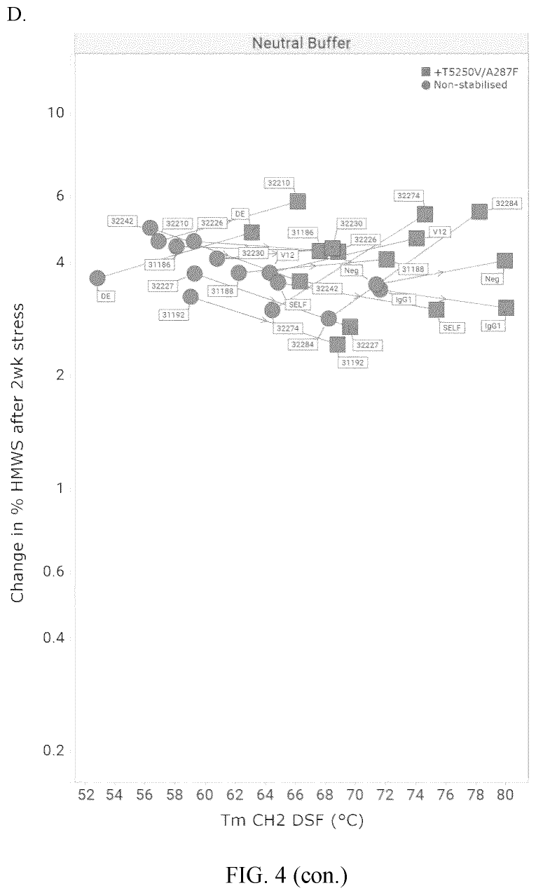

Subsequently, each variant was normalised to 10 mg/ml, dialysed into either acetate or phosphate-based buffers for testing under mildly acidic or neutral conditions, respectively, and incubated at either 4° C. or 40° C. for 2 weeks. Variants were then each assessed for aggregate, monomer and fragment proportions by size exclusion chromatography, comparing the 4° C. and 40° C. samples. The results are shown in Table 9.3 and FIG. 4.

TABLE 9.1
Mutations and Initial Purity of Antibody Variants

| | | | Non-Stabilized | | | +T250V/A287F | | |
|---|---|---|---|---|---|---|---|---|
| | | | % | % | % | % | % | % |
| | CH2 Domain Mutations | | Total | Total | Total | Total | Total | Total |
| Variant | Chain A | Chain B | HMW | Monomer | LMW | HMW | Monomer | LMW |
| Wild- | — | — | 0.37 | 99.63 | 0 | 0.73 | 99.27 | 0 |
| Type | | | | | | | | |
| IgG1 | | | | | | | | |
| Negative | L234A L235A | L234A L235A D265S | 0.52 | 99.48 | 0 | 0.89 | 99.11 | 0 |
| Control | D265S | | | | | | | |
| v12¹ | E233D G237D | E233D G237D P238D H268D | 0.59 | 99.41 | 0 | 0.54 | 99.46 | 0 |
| | P238D H268D | P271G A330R | | | | | | |
| | P271G A330R | | | | | | | |
| SELF² | S267E L328F | S267E L328F | 0.71 | 99.29 | 0 | 0.66 | 99.34 | 0 |
| DE³ | S239D I332E | S239D I332E | 4.2 | 95.8 | 0 | 0.3 | 99.7 | 0 |
| 31186 | G236N G237D | G236D G237F S239D S267V | 0.75 | 99.25 | 0 | 0.56 | 99.44 | 0 |
| | | H268D + Template 1.1⁴ | | | | | | |
| 32210 | G236N G237D | G236D G237F S239D S267V | 0.55 | 99.45 | 0 | 0.36 | 99.64 | 0 |
| | | H268D I332L + Template 1.1⁴ | | | | | | |
| 32242 | G236N G237A | G236D G237F S239D S267V | 0.42 | 99.58 | 0 | 0.42 | 99.58 | 0 |
| | S239P | H268D I332L + Template 1.1⁴ | | | | | | |
| 32226 | L235D G236N | G236D G237F S239D S267V | 0.58 | 99.42 | 0 | 0.58 | 99.42 | 0 |
| | G237A | H268D I332L + Template 1.1⁴ | | | | | | |
| 31192 | L234F L235D | G236D G237F S239D S267V | 0.54 | 99.46 | 0 | 0.42 | 99.58 | 0 |
| | G236N H268Q | H268D + Template 1.1⁴ | | | | | | |
| | A327G A330K | | | | | | | |
| | P331S | | | | | | | |
| 31188 | L235F G236N | G236D G237F S239D S267V | 0.34 | 99.66 | 0 | 0.65 | 99.35 | 0 |
| | G237A | H268D + Template 1.2⁵ | | | | | | |
| 32230 | L235V G236N | G236D G237F S239D S267V | 0.45 | 99.55 | 0 | 0.7 | 99.3 | 0 |
| | G237A | H268D I332L + Template 1.1⁴ | | | | | | |
| 32227 | L235E G236N | G236D G237F S239D S267V | 0.39 | 99.61 | 0 | 0.56 | 99.44 | 0 |
| | G237A | H268D I332L + Template 1.1⁴ | | | | | | |
| 32284 | L235D G236N | G236D G237F S239D S267V | 0.81 | 99.19 | 0 | 1.06 | 98.94 | 0 |
| | G237A | H268D + Template 7.16 | | | | | | |
| 32274 | L234F G236N | G236D G237L S239D V266L | 0.69 | 99.31 | 0 | 0.74 | 99.26 | 0 |
| | H268Q A327G | S267A H268D | | | | | | |
| | P329A A330K | | | | | | | |
| | P331S | | | | | | | |

¹Mimoto, et al., 2013, Prot. Eng. Des. Sel., 26: 589-598
²Chu, et al., 2008, Mol. Immunol., 45: 3926-3933
³Lazar, et al., 2006, PNAS, 103: 4005-4010
⁴“Template 1.1” indicates indicates a replacement of the amino acid residues at positions 325-331 in Chain B with the sequence: STWFIGGYAT [SEQ ID NO: 3]
⁵“Template 1.2” indicates indicates a replacement of the amino acid residues at positions 325-331 in Chain B with the sequence: STWFDKGYAT [SEQ ID NO: 5]
⁶“Template 7.1” indicates indicates a replacement of the amino acid residues at positions 325-331 in Chain B with the sequence: GLDHRGKGYV [SEQ ID NO: 4]

TABLE 9.2
Thermal Stability of Parental and Stabilized Variants

| | CH2 Tm (° C.) | | |
|---|---|---|---|
| Variant | Non-Stabilized | +T250V/A287F | Δ CH2 Tm (° C.) |
| Wild-Type IgG1 | 71.6 | 80.0 | +8.4 |
| Negative Control | 71.4 | 79.9 | +8.5 |
| v12 | 64.3 | 74.0 | +9.8 |
| SELF | 64.8 | 75.4 | +10.6 |
| DE | 52.8 | 63.1 | +10.2 |
| 31186 | 58.1 | 67.6 | +9.5 |
| 32210 | 56.9 | 66.1 | +9.2 |
| 32242 | 56.3 | 66.3 | +9.9 |
| 32226 | 59.2 | 68.8 | +9.6 |
| 31192 | 59.0 | 68.8 | +9.8 |
| 31188 | 62.2 | 72.0 | +9.8 |
| 32230 | 60.8 | 68.5 | +7.7 |
| 32227 | 59.3 | 69.6 | +10.3 |
| 32284 | 68.2 | 78.3 | +10.1 |
| 32274 | 64.4 | 74.6 | +10.2 |

TABLE 9.3
Change in Aggregation Propensity of Parental and Stabilized
Variants after Stress under Neutral and Acidic Conditions

| | Neutral Buffer | | | Acidic Buffer | | |
|---|---|---|---|---|---|---|
| | Δ HMW | | | Δ HMW | | |
| | Species (%)¹ | | ΔΔ | Species (%)¹ | | ΔΔ |
| | Non- | Stabilized | HMW | Non- | Stabilized | HMW |
| | Stabil- | (+T250V/ | Species | Stabil- | (+T250V/ | Species |
| Variant | ized | A287F) | (%)² | ized | A287F) | (%)² |
| Wild- | 3.4 | 3.0 | −0.4 | 0.3 | −0.1 | −0.5 |
| Type | | | | | | |
| IgG1 | | | | | | |
| Negative | 3.5 | 4.0 | +0.6 | −0.1 | 0.2 | +0.3 |
| Control | | | | | | |
| v12 | 3.8 | 4.6 | +0.9 | 1.3 | 1.0 | −0.3 |
| SELF | 3.5 | 3.0 | −0.5 | 0.6 | 0.4 | −0.1 |
| DE | 3.6 | 4.8 | +1.2 | −0.3 | −0.1 | +0.2 |
| 31186 | 4.4 | 4.3 | −0.1 | 10.8 | 1.3 | −9.5 |
| 32210 | 4.6 | 5.8 | +1.3 | 11.9 | 1.1 | −10.7 |
| 32242 | 4.9 | 3.6 | −1.4 | 8.4 | 1.0 | −7.4 |
| 32226 | 4.6 | 4.3 | −0.3 | 3.3 | 1.0 | −2.3 |
| 31192 | 3.2 | 2.4 | −0.8 | 3.8 | 0.9 | −2.9 |
| 31188 | 3.8 | 4.1 | +0.3 | 0.6 | 0.7 | +0.2 |
| 32230 | 4.1 | 4.4 | +0.3 | 1.9 | 0.8 | −1.1 |
| 32227 | 3.7 | 2.7 | −1.1 | 2.8 | 0.6 | −2.2 |
| 32284 | 2.8 | 5.5 | +2.6 | 1.0 | 0.5 | −0.5 |
| 32274 | 3.0 | 5.4 | +2.4 | 0.5 | 0.3 | −0.2 |

¹Change in total HMW species after 2-week incubation at 40° C. in indicated buffer
²Δ HMW species [stabilized] − Δ HMW species [non-stabilized]

As can be seen from Table 9.2, the incorporation of the T250V/A287F stability-enhancing mutations successfully increased the thermal stability of the fifteen variants by between 7.7° C. and 10.6° C. Thus, these stability-enhancing mutations are capable of increasing CH2 stability independent of the starting stability of the antibody construct.

The results in Table 9.3 show that after the 2-week incubation at 40° C. under the acidic conditions, three of the variants with the lowest CH2 Tm (variants v31186, v32210 and v32242) showed high levels of observed aggregation by analytical SEC. When the stability-enhancing T250V/A287F mutations were added to these variants, the Tm of the variants increased and minimal amounts of HMW species were detected after the stress experiment. The strong and approximately exponential correlation between CH2 Tm and observed aggregation under mildly acidic conditions for these variants can also be seen in FIG. 4. Little change in high molecular weight species (<2%) was observed for variants having an initial (non-stabilized) CH2 Tm above 60° C. Small changes in high molecular weight species observed following incubation under neutral conditions appears independent of the CH2 Tm.

Overall, these results indicate that incorporation of stabilizing mutations generally, and T250V/A287F specifically, can lower aggregation propensity under mildly acidic conditions.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

1.-4. (canceled)

5. An Fc variant comprising from one to three stability-enhancing amino acid mutations, the mutations comprising:
(a) one or more mutation selected from: a mutation at position 287 which is a substitution with Phe, His, Met, Trp or Tyr; a mutation at position 308 which is a substitution with Ile, and a mutation at position 309 which is a substitution with Gln or Thr, or
(b) two or more mutations selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr; a mutation at position 428 which is a substitution with Phe, and a pair of mutations at position 242 and position 336 which are both substitutions with Cys, or
(c) three or more mutations comprising: a pair of mutations at position 242 and position 336 which are both substitutions with Cys, and a mutation selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr, and a mutation at position 428 which is a substitution with Phe,
wherein the Fc variant has an increased CH2 domain melting temperature (Tm) as compared to a parental Fc that does not include the one or more stability-enhancing amino acid mutations, and
wherein the numbering of amino acids is according to the EU index.

6. The Fc variant according to claim 5 comprising a mutation at position 287 which is a substitution with Phe, His, Met, Trp or Tyr.

7. The Fc variant according to claim 6, wherein the mutation at position 287 is a substitution with Phe.

8. The Fc variant according to claim 5 comprising a mutation at position 308 which is a substitution with Ile.

9. The Fc variant according to claim 5 comprising a mutation at position 309 which is a substitution with Gln or Thr.

10. The Fc variant according to claim 9, wherein the mutation at position 309 is a substitution with Gln.

11. The Fc variant according to claim 5 comprising a mutation at position 250 which is a substitution with Ala, Ile or Val, and a mutation at position 287 which is a substitution with Phe, His, Met, Trp or Tyr.

12. The Fc variant according to claim 11, wherein the mutation at position 250 is a substitution with Val.

13. The Fc variant according to claim 12, wherein the mutation at position 287 is a substitution with Phe.

14. The Fc variant according to claim 5 comprising a mutation at position 250 which is a substitution with Ala, Ile or Val, and a mutation at position 309 which is a substitution with Gln or Thr.

15. The Fc variant according to claim 14, wherein the mutation at position 250 is a substitution with Val.

16. The Fc variant according to claim 15, wherein the mutation at position 309 is a substitution with Gln.

17. The Fc variant according to claim 5 comprising a mutation at position 250 which is a substitution with Ala, Ile or Val, and a mutation at position 428 which is a substitution with Phe.

18. The Fc variant according to claim 17, wherein the mutation at position 250 is a substitution with Val.

19. The Fc variant according to claim 5 comprising a mutation at position 287 which is a substitution with Phe, His, Met, Trp or Tyr, and a mutation at position 428 which is a substitution with Phe.

20. The Fc variant according to claim 19, wherein the mutation at position 287 is a substitution with Phe.

21. The Fc variant according to claim 5 comprising a pair of mutations at position 242 and position 336 which are both substitutions with Cys.

22. The Fc variant according to claim 5 comprising a pair of mutations at position 242 and position 336 which are both substitutions with Cys, and a mutation at position 308 which is a substitution with Ile.

23. The Fc variant according to claim 5, wherein the stability-enhancing mutations comprised by the Fc variant are selected from: 250V, 287F, 308I, 309Q, 428F, 242C_336C, 287F/428F, 250V/287F, 250V/309Q, 250V/428F and 242C_336C/308I.

24. The Fc variant according to claim 5, wherein the stability-enhancing mutations comprised by the Fc variant are selected from: 287F/428F, 250V/287F, 250V/309Q, 250V/428F and 242C_336C/308I.

25. The Fc variant according to claim 5, wherein the Fc variant is based on an IgG, IgA, IgD, IgE or IgM Fc.

26. The Fc variant according to claim 25, wherein the Fc variant is based on a human IgG, IgA, IgD, IgE or IgM Fc.

27. The Fc variant according to claim 5, wherein the Fc variant is based on an IgG Fc.

28. The Fc variant according to claim 27, wherein the IgG Fc is an IgG1 Fc.

29. The Fc variant according to claim 27, wherein the IgG Fc is a human IgG Fc.

30. The Fc variant according to claim 5, wherein the parental Fc comprises one or more amino acid mutations that improve a function of the Fc region.

31. The Fc variant according to claim 5, wherein the parental Fc comprises one or more amino acid mutations that improve a function of the Fc region and decrease the CH2 domain Tm of the corresponding wild-type Fc.

32. The Fc variant according to claim 5, wherein the CH2 domain Tm of the Fc variant is increased by at least 0.5° C. as compared to the parental Fc.

33. The Fc variant according to claim 32, wherein the CH2 domain Tm of the Fc variant is increased by at least 1.0° C., at least 2.0° C., or at least 3.0° C., as compared to the parental Fc.

34. The Fc variant according to claim 5, wherein the CH2 domain Tm of the Fc variant is increased by between 0.5° C. and 9.0° C. as compared to the parental Fc.

35. The Fc variant according to claim 5, wherein the CH2 domain Tm of the Fc variant is increased by between 2.0° C. and 10.5° C. as compared to the parental Fc.

36. A polypeptide comprising the Fc variant according to claim 5 and one or more proteinaceous moieties fused or covalently attached to the Fc variant.

37. The polypeptide according to claim 36, wherein the one or more proteinaceous moieties comprise an antigen-binding domain, a ligand, a receptor, a receptor fragment, a cytokine or an antigen.

38. The polypeptide according to claim 37, wherein at least one of the one or more proteinaceous moieties is an antigen-binding domain.

39. The polypeptide according to claim 37, wherein the antigen-binding domain is a Fab or scFv.

40. The polypeptide according to claim 36, wherein the polypeptide is an antibody or an antigen-binding antibody fragment.

41. The polypeptide according to claim 40, wherein the polypeptide is a therapeutic antibody or antibody fragment.

42. A polynucleotide or set of polynucleotides encoding the Fc variant according to claim 5.

43. A polynucleotide or set of polynucleotides encoding the polypeptide according to claim 36.

44. A vector or set of vectors comprising one or more polynucleotides encoding the polypeptide according to claim 36.

45. A host cell comprising one or more polynucleotides encoding the polypeptide according to claim 36.

46. (canceled)

47. A method of preparing the polypeptide according to claim 36 comprising transfecting a host cell with one or more polynucleotides encoding the polypeptide, and culturing the host cell under conditions suitable for expression of the polypeptide.

48. A pharmaceutical composition comprising the polypeptide according to claim 36.

49.-52. (canceled)

53. A method of increasing the CH2 domain melting temperature (Tm) of an Fc comprising introducing into a parental Fc one to three stability-enhancing amino acid mutations to provide an Fc variant having an increased CH2 domain Tm as compared to the parental Fc, the mutations comprising:
(a) one or more mutation selected from: a mutation at position 287 which is a substitution with Phe, His, Met, Trp or Tyr; a mutation at position 308 which is a substitution with Ile, and a mutation at position 309 which is a substitution with Gln or Thr, or
(b) two or more mutations selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr; a mutation at position 428 which is a substitution with Phe, and a pair of mutations at position 242 and position 336 which are both substitutions with Cys, or
(c) three or more mutations comprising: a pair of mutations at position 242 and position 336 which are both substitutions with Cys, and a mutation selected from: a mutation at position 250 which is a substitution with Ala, Ile or Val; a mutation at position 287 which is a substitution with Phe, His, Met, Trp or Tyr; a mutation at position 308 which is a substitution with Ile; a mutation at position 309 which is a substitution with Gln or Thr, and a mutation at position 428 which is a substitution with Phe,
wherein the numbering of amino acids is according to the EU index.

54. The method according to claim 53, wherein the CH2 domain Tm of the Fc variant is increased by at least 0.5° C. as compared to the parental Fc.

55. The method according to claim 54, wherein the CH2 domain Tm of the Fc variant is increased by at least 1.0° C., at least 2.0° C., or at least 3.0° C., as compared to the parental Fc.

56. The method according to claim 53, wherein the CH2 domain Tm of the Fc variant is increased by between 0.5° C. and 9.0° C. as compared to the parental Fc.

57. The method according to claim 53, wherein the CH2 domain Tm of the Fc variant is increased by between 2.0° C. and 10.5° C. as compared to the parental Fc.

58. The method according to claim 53, wherein introducing the stability-enhancing amino acid mutations into the parental Fc provides an Fc variant showing decreased aggregation under mildly acidic conditions as compared to the parental Fc region.

59. The method according to claim 58, wherein the stability-enhancing amino acid mutations comprise 250V and 287F.
